SEQUENCE LISTING (110 sequences)

SEQ ID NO: 1 - DNA, 21 bp, synthetic construct
  source 1..21: mol_type = other DNA
cgtctgggctttgtggatgtg

SEQ ID NO: 2 - DNA, 21 bp, synthetic construct
  source 1..21: mol_type = other DNA
tgacctgaaggaagggcagtg

SEQ ID NO: 3 - DNA, 20 bp, synthetic construct
  source 1..20: mol_type = other DNA
ggaaatggctcggggacaaa

SEQ ID NO: 4 - DNA, 20 bp, synthetic construct
  source 1..20: mol_type = other DNA
ctagccccagcaacacagtt

SEQ ID NO: 5 - DNA, 20 bp, synthetic construct
  source 1..20: mol_type = other DNA
acacatcccaagagccacag

SEQ ID NO: 6 - DNA, 20 bp, synthetic construct
  source 1..20: mol_type = other DNA
acccttattcatgcggggac

SEQ ID NO: 7 - DNA, 20 bp, synthetic construct
  source 1..20: mol_type = other DNA
ctagagccggcaagacatgg

SEQ ID NO: 8 - DNA, 20 bp, synthetic construct
  source 1..20: mol_type = other DNA
ctggcatctacatggtcggg

SEQ ID NO: 9 - DNA, 20 bp, synthetic construct
  source 1..20: mol_type = other DNA
taaagtcctccaccctcgtc

SEQ ID NO: 10 - DNA, 20 bp, synthetic construct
  source 1..20: mol_type = other DNA
ctagtcagttgccttgtgcc

SEQ ID NO: 11 - DNA, 23 bp, synthetic construct
  source 1..23: mol_type = other DNA
gcttcttctgtaggtctgatggt

SEQ ID NO: 12 - DNA, 20 bp, synthetic construct
  source 1..20: mol_type = other DNA
taggcgaagggacagaaacc

SEQ ID NO: 13 - DNA, 20 bp, synthetic construct
  source 1..20: mol_type = other DNA
cccatcagcatcagcactac

SEQ ID NO: 14 - DNA, 24 bp, synthetic construct
  source 1..24: mol_type = other DNA
tggttcaaatataacatccttgcc

SEQ ID NO: 15 - DNA, 20 bp, synthetic construct
  source 1..20: mol_type = other DNA
gcaaacccgttttgttcgca

SEQ ID NO: 16 - DNA, 20 bp, synthetic construct
  source 1..20: mol_type = other DNA
cactgatagctgcccctctt

SEQ ID NO: 17 - DNA, 20 bp, synthetic construct
  source 1..20: mol_type = other DNA
gaagcccgggatcggattag

SEQ ID NO: 18 - DNA, 20 bp, synthetic construct
  source 1..20: mol_type = other DNA
tcagcaactcctttcgggtc

SEQ ID NO: 19 - DNA, 21 bp, synthetic construct
  source 1..21: mol_type = other DNA
gcagctttgttaccggattgg

SEQ ID NO: 20 - DNA, 21 bp, synthetic construct
  source 1..21: mol_type = other DNA
acgggcttaaatggagaagca

SEQ ID NO: 21 - DNA, 23 bp, synthetic construct
  source 1..23: mol_type = other DNA
ttaaaaacctggatcggaaccaa

SEQ ID NO: 22 - DNA, 22 bp, synthetic construct
  source 1..22: mol_type = other DNA
gcattagcttcagatttacggg

SEQ ID NO: 23 - DNA, 28 bp, synthetic construct
  source 1..28: mol_type = other DNA
gcctattatctataccctccagcacatc

SEQ ID NO: 24 - DNA, 24 bp, synthetic construct
  source 1..24: mol_type = other DNA
tccatctcccatcctccacatcag

SEQ ID NO: 25 - DNA, 22 bp, synthetic construct
  source 1..22: mol_type = other DNA
tccattgaggtggagagctttc

SEQ ID NO: 26 - DNA, 23 bp, synthetic construct
  source 1..23: mol_type = other DNA
ctacaggctccgagatgaacaac

SEQ ID NO: 27 - DNA, 25 bp, synthetic construct
  source 1..25: mol_type = other DNA
agaccctcacactcagatcatcttc

SEQ ID NO: 28 - DNA, 20 bp, synthetic construct
  source 1..20: mol_type = other DNA
cctccacttggtggtttgct

SEQ ID NO: 29 - DNA, 20 bp, synthetic construct
  source 1..20: mol_type = other DNA
ccttatcatcccggccagtc

SEQ ID NO: 30 - DNA, 20 bp, synthetic construct
  source 1..20: mol_type = other DNA
gtcccaccccacctatcaag

SEQ ID NO: 31 - DNA, 20 bp, synthetic construct
  source 1..20: mol_type = other DNA
cgctcttcacgaaatccagc

SEQ ID NO: 32 - DNA, 20 bp, synthetic construct
  source 1..20: mol_type = other DNA
tcatgttgtggggctttgct

SEQ ID NO: 33 - DNA, 20 bp, synthetic construct
  source 1..20: mol_type = other DNA
cttgccttgtacgcaactcc

SEQ ID NO: 34 - DNA, 20 bp, synthetic construct
  source 1..20: mol_type = other DNA
gcactgccaccagagaattg

SEQ ID NO: 35 - DNA, 20 bp, synthetic construct
  source 1..20: mol_type = other DNA
gccctcagcatgatggactt

SEQ ID NO: 36 - DNA, 21 bp, synthetic construct
  source 1..21: mol_type = other DNA
tgtcttgcctccaaaggatgt

SEQ ID NO: 37 - DNA, 20 bp, synthetic construct
  source 1..20: mol_type = other DNA
atcgcagggagggtcatttg

SEQ ID NO: 38 - DNA, 20 bp, synthetic construct
  source 1..20: mol_type = other DNA
tggggtcaagagggtcagaa

SEQ ID NO: 39 - DNA, 16 bp, synthetic construct
  source 1..16: mol_type = other DNA
gagtccgcagcaggtg

SEQ ID NO: 40 - DNA, 18 bp, synthetic construct
  source 1..18: mol_type = other DNA
gtgtcagagtccatggga

SEQ ID NO: 41 - DNA, 20 bp, synthetic construct
  source 1..20: mol_type = other DNA
gacaagctgagcgacgagta

SEQ ID NO: 42 - DNA, 19 bp, synthetic construct
  source 1..19: mol_type = other DNA
gtcagctccagcaccttgt

SEQ ID NO: 43 - DNA, 20 bp, synthetic construct
  source 1..20: mol_type = other DNA
atgcgggacatgtttgatga

SEQ ID NO: 44 - DNA, 21 bp, synthetic construct
  source 1..21: mol_type = other DNA
tcggatgaggatgctgaacac

SEQ ID NO: 45 - DNA, 20 bp, synthetic construct
  source 1..20: mol_type = other DNA
tggcagctcttctcaaagca

SEQ ID NO: 46 - DNA, 21 bp, synthetic construct
  source 1..21: mol_type = other DNA
cccaagagtcgtccaggtcat

SEQ ID NO: 47 - DNA, 20 bp, synthetic construct
  source 1..20: mol_type = other DNA
gccctcagcatgatggactt

SEQ ID NO: 48 - DNA, 21 bp, synthetic construct
  source 1..21: mol_type = other DNA
tgtcttgcctccaaaggatgt

SEQ ID NO: 49 - DNA, 20 bp, synthetic construct
  source 1..20: mol_type = other DNA
agctggttgaagtgtccagt

SEQ ID NO: 50 - DNA, 21 bp, synthetic construct
  source 1..21: mol_type = other DNA
gcaggttaggaaatagcgaca

SEQ ID NO: 51 - DNA, 20 bp, synthetic construct
  source 1..20: mol_type = other DNA
ggttccctggacgttatccg

SEQ ID NO: 52 - DNA, 20 bp, synthetic construct
  source 1..20: mol_type = other DNA
ggcggaggatgaagagttcc

SEQ ID NO: 53 - DNA, 20 bp, synthetic construct
  source 1..20: mol_type = other DNA
cgctcccgacccatctttag

SEQ ID NO: 54 - DNA, 20 bp, synthetic construct
  source 1..20: mol_type = other DNA
actgggctacatcctcgact

SEQ ID NO: 55 - DNA, 20 bp, synthetic construct
  source 1..20: mol_type = other DNA
accagcagcctaaaagcagt

SEQ ID NO: 56 - DNA, 20 bp, synthetic construct
  source 1..20: mol_type = other DNA
cccagagagagcgtagtgtt

SEQ ID NO: 57 - DNA, 20 bp, synthetic construct
  source 1..20: mol_type = other DNA
caggccgagaaggagaagct

SEQ ID NO: 58 - DNA, 20 bp, synthetic construct
  source 1..20: mol_type = other DNA
ggctcgcagatcagcagact

SEQ ID NO: 59 - DNA, 20 bp, synthetic construct
  source 1..20: mol_type = other DNA
gtacctgcgggacagcttag

SEQ ID NO: 60 - DNA, 20 bp, synthetic construct
  source 1..20: mol_type = other DNA
tcaggtcatgttggaaacca

SEQ ID NO: 61 - DNA, 23 bp, synthetic construct
  source 1..23: mol_type = other DNA
ggcgcagcaggtgtatactatgt

SEQ ID NO: 62 - DNA, 21 bp, synthetic construct
  source 1..21: mol_type = other DNA
caaacaccggaatccatacgt

SEQ ID NO: 63 - DNA, 23 bp, synthetic construct
  source 1..23: mol_type = other DNA
cgcaagctgaagaaatatgacaa

SEQ ID NO: 64 - DNA, 19 bp, synthetic construct
  source 1..19: mol_type = other DNA
tcgatcctggccacatctc

SEQ ID NO: 65 - DNA, 20 bp, synthetic construct
  source 1..20: mol_type = other DNA
aagggaaggacatcaagggg

SEQ ID NO: 66 - DNA, 20 bp, synthetic construct
  source 1..20: mol_type = other DNA
ggtgaactcattgcggacca

SEQ ID NO: 67 - DNA, 20 bp, synthetic construct
  source 1..20: mol_type = other DNA
gtcaccatccggtcagagag

SEQ ID NO: 68 - DNA, 20 bp, synthetic construct
  source 1..20: mol_type = other DNA
cctgtcgtctgcggtgattt

SEQ ID NO: 69 - DNA, 20 bp, synthetic construct
  source 1..20: mol_type = other DNA
acgggaaggagagcacgata

SEQ ID NO: 70 - DNA, 20 bp, synthetic construct
  source 1..20: mol_type = other DNA
agccctcattgcaccttctc

SEQ ID NO: 71 - DNA, 27 bp, synthetic construct
  source 1..27: mol_type = other DNA
atacagagttcgttatctagccaagga

SEQ ID NO: 72 - DNA, 22 bp, synthetic construct
  source 1..22: mol_type = other DNA
ccattgggctgtacaaaagaca

SEQ ID NO: 73 - DNA, 21 bp, synthetic construct
  source 1..21: mol_type = other DNA
cccttctccttggccatctat

SEQ ID NO: 74 - DNA, 22 bp, synthetic construct
  source 1..22: mol_type = other DNA
gagtcgattggcactgaaaatg

SEQ ID NO: 75 - DNA, 20 bp, synthetic construct
  source 1..20: mol_type = other DNA
gggacaacacgcatttcatg

SEQ ID NO: 76 - DNA, 21 bp, synthetic construct
  source 1..21: mol_type = other DNA
gttgcttgcagactgggacat

SEQ ID NO: 77 - DNA, 21 bp, synthetic construct
  source 1..21: mol_type = other DNA
acagtccggaaaggactgaca

SEQ ID NO: 78 - DNA, 21 bp, synthetic construct
  source 1..21: mol_type = other DNA
cactccacgtagcagcaaggt

SEQ ID NO: 79 - DNA, 20 bp, synthetic construct
  source 1..20: mol_type = other DNA
ccatcagaacagcgcaagaa

SEQ ID NO: 80 - DNA, 20 bp, synthetic construct
  source 1..20: mol_type = other DNA
actgggctacatcctcgact

SEQ ID NO: 81 - DNA, 20 bp, synthetic construct
  source 1..20: mol_type = other DNA
ctgaagcagatccgcagctt

SEQ ID NO: 82 - DNA, 21 bp, synthetic construct
  source 1..21: mol_type = other DNA
ggtgagatgtgctgggtcatg

SEQ ID NO: 83 - DNA, 20 bp, synthetic construct
  source 1..20: mol_type = other DNA
tggagtccatcttcctgtcc

SEQ ID NO: 84 - DNA, 23 bp, synthetic construct
  source 1..23: mol_type = other DNA
cgaatatggtcgtttctgaaagt

SEQ ID NO: 85 - DNA, 26 bp, synthetic construct
  source 1..26: mol_type = other DNA
gaagaagtgtctggactgtgtcattt

SEQ ID NO: 86 - DNA, 24 bp, synthetic construct
  source 1..24: mol_type = other DNA
ttaattgtgggatcaggagagcat

SEQ ID NO: 87 - DNA, 19 bp, synthetic construct
  source 1..19: mol_type = other DNA
tcgtggtggtgggtgtgat

SEQ ID NO: 88 - DNA, 20 bp, synthetic construct
  source 1..20: mol_type = other DNA
gggtccgattgatctttgca

SEQ ID NO: 89 - DNA, 20 bp, synthetic construct
  source 1..20: mol_type = other DNA
cggagcagggtattggaaca

SEQ ID NO: 90 - DNA, 20 bp, synthetic construct
  source 1..20: mol_type = other DNA
ctgagggctgaaactccagg

SEQ ID NO: 91 - DNA, 20 bp, synthetic construct
  source 1..20: mol_type = other DNA
cttcatcagcaccttggggt

SEQ ID NO: 92 - DNA, 20 bp, synthetic construct
  source 1..20: mol_type = other DNA
ggcacaccttgaaaaggcag

SEQ ID NO: 93 - DNA, 20 bp, synthetic construct
  source 1..20: mol_type = other DNA
gcttcatcagcactttgggg

SEQ ID NO: 94 - DNA, 20 bp, synthetic construct
  source 1..20: mol_type = other DNA
atggggcacaccttgaagag

SEQ ID NO: 95 - DNA, 20 bp, synthetic construct
  source 1..20: mol_type = other DNA
aagcttaaagcctccagccg

SEQ ID NO: 96 - DNA, 19 bp, synthetic construct
  source 1..19: mol_type = other DNA
gtgggtagtcgtggtcagc

SEQ ID NO: 97 - DNA, 20 bp, synthetic construct
  source 1..20: mol_type = other DNA
atgggatggagagcctgagt

SEQ ID NO: 98 - DNA, 20 bp, synthetic construct
  source 1..20: mol_type = other DNA
ggtactggtactgcgtctcc

SEQ ID NO: 99 - DNA, 20 bp, synthetic construct
  source 1..20: mol_type = other DNA
ggtgtgaagaagggggatgg

SEQ ID NO: 100 - DNA, 20 bp, synthetic construct
  source 1..20: mol_type = other DNA
ctaggctggcattcgcacta

SEQ ID NO: 101 - DNA, 20 bp, synthetic construct
  source 1..20: mol_type = other DNA
gtgggagctggtgatagagc

SEQ ID NO: 102 - DNA, 20 bp, synthetic construct
  source 1..20: mol_type = other DNA
tcaccttcctcaaaccaggc

SEQ ID NO: 103 - DNA, 20 bp, synthetic construct
  source 1..20: mol_type = other DNA
ttgctcgagttgaaccctcc

SEQ ID NO: 104 - DNA, 20 bp, synthetic construct
  source 1..20: mol_type = other DNA
cggctttcttcagagcagga

SEQ ID NO: 105 - DNA, 21 bp, synthetic construct
  source 1..21: mol_type = other DNA
ttgtgtctcccttgtccatgc

SEQ ID NO: 106 - DNA, 22 bp, synthetic construct
  source 1..22: mol_type = other DNA
gggtcaaatccctctgagatcc

SEQ ID NO: 107 - DNA, 20 bp, synthetic construct
  source 1..20: mol_type = other DNA
aagcttttcttgctggcgtg

SEQ ID NO: 108 - DNA, 20 bp, synthetic construct
  source 1..20: mol_type = other DNA
ctcccaagcacatctacgca

SEQ ID NO: 109 - DNA, 20 bp, synthetic construct
  source 1..20: mol_type = other DNA
gaggaggaggaggtggagtt

SEQ ID NO: 110 - DNA, 20 bp, synthetic construct
  source 1..20: mol_type = other DNA
tccctcttggtcaaagtgct

(19) United States
(12) Patent Application Publication
(10) Pub. No.: US 2026/0263425 A1
(43) Pub. Date: Sep. 10, 2026

(54) USE OF COMPOSITION CONTAINING CANDESARTAN OR AZILSARTAN

(71) Applicant: GACHON UNIVERSITY OF INDUSTRY-ACADEMIC COOPERATION FOUNDATION, Seongnam-si, Gyeonggi-do (KR)

(72) Inventors: Byung-Chul OH, Incheon (KR); Jinwook LEE, Incheon (KR); Ok Hee KIM, Incheon (KR); Seung-Yong SEO, Incheon (KR); Yunjae JUNG, Incheon (KR)

(21) Appl. No.: 19/155,239

(22) PCT Filed: Feb. 7, 2024
(86) PCT No.: PCT/KR2024/001784

§ 371 Date: Aug. 8, 2025

(30) Foreign Application Priority Data
Feb. 10, 2023 (KR) 10-2023-0018171

(51) Int. Cl. A61K 31/4184; A61K 31/4245; A61P 1/16; A61P 3/06; A61P 3/10; A61P 5/50; A61P 13/12; A61P 19/06

(52) U.S. Cl. CPC A61K 31/4184; A61K 31/4245; A61P 1/16; A61P 3/06; A61P 3/10; A61P 5/50; A61P 13/12; A61P 19/06

(57) ABSTRACT

The present invention relates to a pharmaceutical composition for preventing or treating a metabolic disease, comprising candesartan or azilsartan; a method for preventing or treating a metabolic disease, comprising administering the composition to a subject; a food composition for preventing or improving a metabolic disease, comprising candesartan or azilsartan; and a feed composition.

### CROSS-REFERENCE TO RELATED APPLICATIONS

The present application is a 35 U.S.C. § 371 national-phase filing of International Application No. PCT/KR2024/001784, filed on Feb. 7, 2024, which claims the benefit of Korean Patent Application No. 10-2023-0018171, filed on Feb. 10, 2023, both of which applications are incorporated herein by reference in their entirety.

### TECHNICAL FIELD

The present invention relates to a pharmaceutical composition for preventing or treating a metabolic disease, comprising candesartan or azilsartan; a method for preventing or treating a metabolic disease, comprising administering the composition to a subject; a food composition for preventing or improving a metabolic disease, comprising candesartan or azilsartan; and a feed composition.

### BACKGROUND ART

Angiotensin II receptor blockers (ARBs), or AT1 receptor blockers, are a class of drugs that regulate the renin-angiotensin system. ARBs are primarily used in the treatment of hypertension, but are also used in the treatment of diabetic nephropathy and congestive heart failure. These substances inhibit the activation of angiotensin II-AT1 receptors by blocking the binding of angiotensin II to angiotensin II-AT1 receptors.

ARBs are mainly used in hypertensive patients who are unable to receive ACE inhibitor therapy. The types of ARBs include eprosartan, fimasartan, irbesartan, losartan, olmesartan, telmisartan, valsartan, azilsartan, and candesartan. Among the above, candesartan is also administered to patients with heart failure who are unable to receive ACE inhibitor therapy, and clinical results have been reported that irbesartan and losartan are beneficial to hypertensive patients with type 2 diabetes and delay diabetic nephropathy. Additionally, it has been reported that some ARBs can improve insulin sensitivity through their effects on glucose homeostasis, and these results suggest that some ARBs can attenuate insulin resistance and improve glucose and lipid metabolism.

Nonetheless, it remains a matter of controversy whether angiotensin II receptor blockade attenuates insulin resistance. Furthermore, it is unclear whether such effects result from the inherent properties of ARBs or from other unidentified effects thereof.

Meanwhile, insulin resistance is associated with several pathological signals, including oxidative stress, inflammation, dysregulated intracellular Ca²⁺ homeostasis, endoplasmic reticulum stress, and mitochondrial dysfunction. In particular, increased intracellular Ca²⁺ levels associated with obesity have been shown to be a major pathological symptom of insulin resistance and type 2 diabetes.

Insulin resistance and obesity-related hypertension often occur concomitantly and are components of metabolic syndrome and cardiovascular disease. In particular, intracellular Ca²⁺ overload has been reported as an early symptom of hypertension, arteriosclerosis, and cardiovascular disease. Among the available antihypertensives, verapamil, a Ca²⁺ channel blocker (a common antihypertensive agent that targets L-type Ca²⁺ channels), has been shown to prevent obesity or streptozotocin (STZ)-induced diabetes by promoting functional beta cell mass. It delays the loss of beta cell function and reduces insulin requirements, thereby promoting insulin production in patients with type 1 diabetes. However, high-dose verapamil therapy is unsuitable for use in patients with diabetes or insulin resistance because it causes cardiac side effects.

Recently, it has been reported that excessive intracellular Ca²⁺ induced by obesity blocks membrane targeting of various pleckstrin homology (PH) domains and interferes with insulin signaling by tightly binding to plasma membrane phosphoinositides (PIPs) to form Ca²⁺-PIP (Proceedings of the National Academy of Sciences 114, 11926-11931). Accordingly, excessive intracellular Ca²⁺ is an important negative regulator in insulin signaling. However, despite the above evidence, there is currently no effective therapeutic method targeting insulin resistance caused by obesity-related intracellular Ca²⁺ overload.

### DISCLOSURE

### Technical Problem

The present inventors have made efforts to develop an effective therapeutic method targeting insulin resistance caused by intracellular Ca²⁺ overload. As a result, it was confirmed that, among ARBs, candesartan or azilsartan can significantly improve insulin resistance and, through normalization of intracellular Ca²⁺ homeostasis, serve as a safe and effective therapeutic method for treating obesity-related insulin resistance and metabolic disease, thereby completing the present invention.

### Technical Solution

It is one object of the present invention to provide a pharmaceutical composition for preventing or treating a metabolic disease, comprising candesartan or azilsartan.

It is another object of the present invention to provide a method for preventing or treating a metabolic disease, comprising administering the composition to a subject.

It is still another object of the present invention to provide a food composition for preventing or improving a metabolic disease, comprising candesartan or azilsartan.

It is still another object of the present invention to provide a feed composition for preventing or improving a metabolic disease, comprising candesartan or azilsartan.

It is still another object of the present invention to provide use of a composition comprising candesartan or azilsartan in preventing, improving, or treating a metabolic disease.

It is still another object of the present invention to provide use of candesartan or azilsartan, and a composition comprising the same, for the manufacture of a medicament, food product, or feed for preventing, improving, or treating a metabolic disease.

### Advantageous Effects

The composition for preventing and treating metabolic disease according to the present invention, comprising candesartan or azilsartan, exhibits effects of improving insulin resistance including obesity-induced insulin resistance and improving fatty liver, and thus can be provided as a composition for preventing, improving, or treating metabolic disease including fatty liver disease, dyslipidemia, and gout.

### BRIEF DESCRIPTION OF THE DRAWINGS

FIG. 1 illustrates the ability of candesartan and azilsartan to restore impaired insulin signaling.

FIG. 2 illustrates the ability of candesartan and azilsartan to regulate overloaded intracellular Ca²⁺ levels and lipid accumulation.

FIG. 3 illustrates the ability of candesartan and azilsartan to recover insulin-stimulated membrane localization of an impaired AKT PH domain.

FIG. 4 illustrates the effect of candesartan on improving in vitro insulin resistance.

FIG. 5 illustrates the method by which candesartan inhibits intracellular Ca²⁺ overload.

FIG. 6 illustrates the regulation of intracellular Ca²⁺-related transporter expression by candesartan.

FIG. 7 illustrates the regulation of Ca²⁺ content in the endoplasmic reticulum by candesartan.

FIG. 8 illustrates the effect of candesartan on improving insulin resistance in high-fat diet mice.

FIG. 9 illustrates the effect of candesartan on improving fatty liver and the blood lipid profile analysis results.

FIG. 10 illustrates the effect of candesartan on improving renal function and gout through renal function markers.

FIG. 11 illustrates the results of gene set enrichment analysis (GSEA) resulting from candesartan administration.

FIG. 12 illustrates the analysis results of SOCE-related gene and protein expression by candesartan.

FIG. 13 illustrates the analysis results of gene and protein expression related to ER stress, glucose metabolism, and lipid metabolism by candesartan.

FIG. 14 illustrates the effect of candesartan on improving obesity-induced insulin resistance.

FIG. 15 illustrates the ability of candesartan to modulate AKT PH domain membrane localization in vivo.

### DETAILED DESCRIPTION OF PREFERRED EMBODIMENTS

The present invention will be described in detail as follows. Meanwhile, each description and embodiment described herein may be applied to other descriptions and embodiments, respectively. That is, all combinations of various elements described herein fall within the scope of the present invention. Further, the scope of the present invention is not limited by the specific description below.

One aspect of the present invention for achieving the above objects provides a pharmaceutical composition for preventing or treating a metabolic disease, comprising candesartan or azilsartan.

As used herein, the term “candesartan” is a drug used in the treatment of hypertension and heart failure. It expands blood vessels and reduces body water content by blocking angiotensin II receptors, which constrict blood vessels and increase body water content, thereby lowering blood pressure. Candesartan is classified as an angiotensin II receptor blocker (ARB) as it acts on the renin-angiotensin-aldosterone system (RAAS), which is activated when the amount of blood flowing into the kidneys decreases, by blocking angiotensin II from binding to receptors. Candesartan is used not only for the treatment of essential hypertension, but also for the treatment of heart failure by reducing the burden on the heart. Additionally, ARBs, including candesartan, exhibit efficacy comparable to previously developed angiotensin converting enzyme (ACE) inhibitors but are advantageous in not causing side effects such as dry cough. Therefore, they can be used for patients who cannot be administered ACE inhibitors. Commercially available products include the following: Atacand®, a candesartan single agent; Atacand Plus®, a combination product that includes the diuretic hydrochlorothiazide in addition to candesartan to effectively control blood pressure; and Rocan Duo®, a combination product that includes an antihyperlipidemic drug to additionally treat hyperlipidemia, which commonly occurs in hypertensive patients.

In the present invention, the candesartan may include candesartan cilexetil and candesartan cilexetil related compound G, but is not limited thereto.

As used herein, the term “azilsartan” refers to a drug used in the treatment of essential hypertension. It expands blood vessels and reduces body water content by blocking angiotensin II receptors, which constrict blood vessels and increase body water content, thereby lowering blood pressure. Similar to candesartan, candesartan is classified as an angiotensin II receptor blocker (ARB) as it acts on the renin-angiotensin-aldosterone system (RAAS), which is activated when the amount of blood flowing into the kidneys decreases, by blocking angiotensin II from binding to receptors. Additionally, ARBs, including azilsartan, exhibit efficacy comparable to previously developed ACE inhibitors but are advantageous in not causing side effects such as dry cough. Therefore, they can be used for patients who cannot be administered ACE inhibitors. Commercially available products include Idalbi®, an azilsartan single agent, and Idalbiclo®, a combination product that includes the diuretic chlorthalidone in addition to azilsartan to effectively control blood pressure.

In the present invention, the azilsartan may include azilsartan medoxomil, but is not limited thereto.

As used herein, the term “angiotensin receptor blocker (ARB)”, also referred to as AT1 receptor antagonist, refers to a drug that regulates the renin-angiotensin system. ARBs are primarily used in the treatment of hypertension, but are also used in the treatment of diabetic nephropathy and congestive heart failure. These substances inhibit the activation of angiotensin II-AT1 receptors by blocking the binding of angiotensin II to angiotensin II-AT1 receptors. The ARBs are mainly used in hypertensive patients who are unable to receive ACE inhibitor therapy. The types of ARBs include eprosartan, fimasartan, irbesartan, losartan, olmesartan, telmisartan, valsartan, azilsartan, and candesartan.

In the present invention, the ARBs may be used by purchasing commercially available products or through synthesis, but are not limited thereto.

For the purpose of the present invention, the ARBs may refer to a specific ARB that alleviates insulin resistance through inhibition of lipid accumulation, normalization of intracellular Ca²⁺ homeostasis, and restoration of insulin signaling, and specifically, may refer to candesartan or azilsartan.

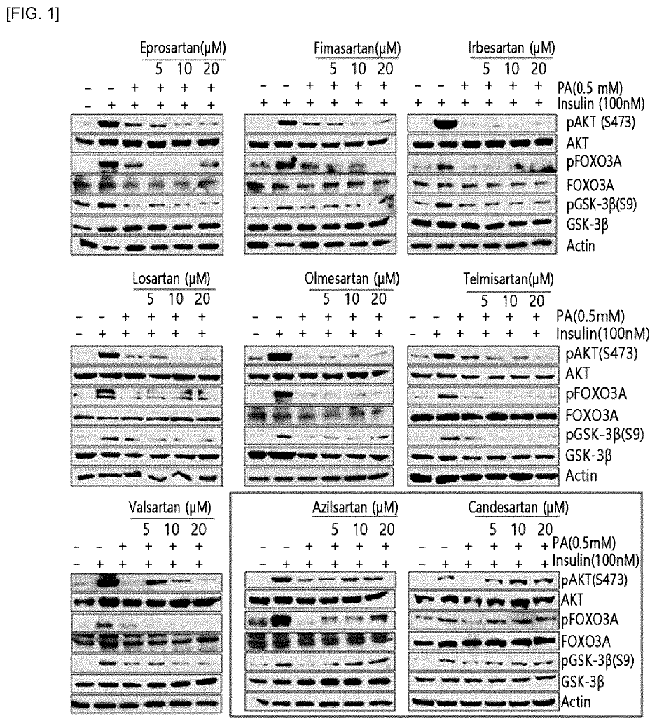

In one embodiment of the present invention, to evaluate whether, and by what mechanism, ARBs restore impaired insulin signaling, the insulin-stimulated phosphorylation of Akt, an important insulin signaling molecule, and of its downstream signaling molecules GSK3β and FOXO3 was investigated. As a result, it was confirmed that, in HepG2 cells treated with palmitic acid, azilsartan and candesartan completely restored the insulin-stimulated phosphorylation of Akt, GSK3B, and FOXO3, respectively (Example 1-1 and FIG. 1).

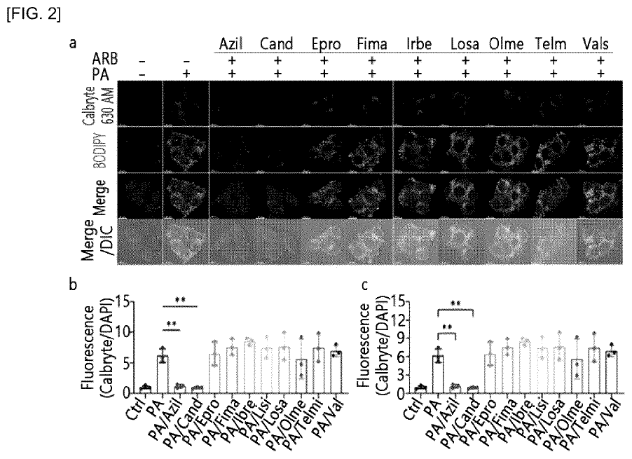

In one embodiment of the present invention, it was confirmed whether ARBs can regulate intracellular Ca²⁺ levels and lipid accumulation in HepG2 cells treated with palmitic acid. As a result, it was confirmed that azilsartan and candesartan significantly reduced the fluorescence intensity for intracellular Ca²⁺ and lipid accumulation in HepG2 cells, whereas eprosartan, fimasartan, irbesartan, losartan, olmesartan, and telmisartan did not significantly affect the intracellular Ca²⁺ concentration or lipid accumulation in HepG2 cells (Example 1-2 and FIG. 2).

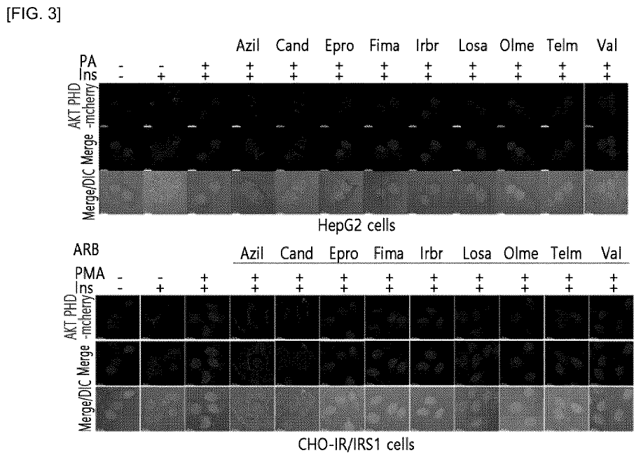

In one embodiment of the present invention, it was evaluated whether ARBs can regulate the intracellular localization of the AKT PH domain that functions as a biosensor for PI(3, 4, 5)P3 using HepG2 or CHO cells treated with palmitic acid. As a result, it was confirmed that attenuation of intracellular Ca²⁺ overload by azilsartan or candesartan restored the impairment of insulin-stimulated membrane localization of the AKT PH domain induced by palmitic acid (Example 1-3 and FIG. 3).

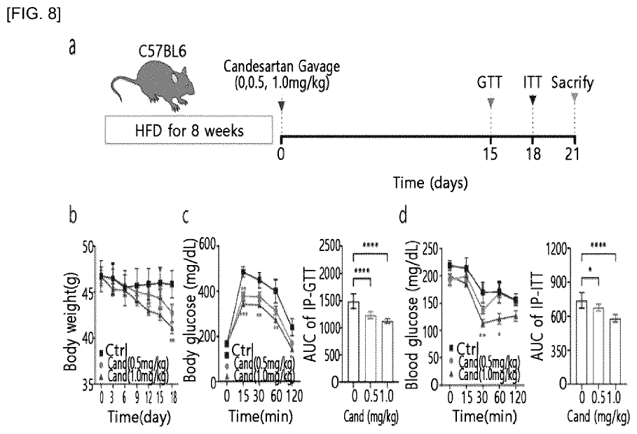

In one embodiment of the present invention, the effect of candesartan cilexetil on improving insulin resistance in high-fat diet mice was confirmed. Through an intraperitoneal glucose tolerance test (IP-GTT) and an intraperitoneal insulin tolerance test (IP-ITT), it was confirmed that the glucose tolerance and insulin sensitivity of candesartan-administered mice were significantly improved, respectively, compared to the control mouse group (Example 5 and FIG. 8).

As used herein, the term “metabolic disease” is a collective term for diseases caused by metabolic disorders in a living body. Specifically, the metabolic disease may be, but is not limited to, a disease that exhibits one or more of the following phenomena, or has the following phenomena or symptoms resulting therefrom as a prodromal syndrome: changes in the function of the intestinal barrier, changes in the blood LPS concentration, occurrence of intestinal inflammation, changes in intestinal mucus, increased insulin resistance, decreased insulin sensitivity, increased fasting blood glucose, increased insulin tolerance, increased glucose tolerance, and increased concentrations of blood creatinine, blood urea nitrogen (BUN), uric acid, and creatine kinase.

The metabolic disease may be specifically selected from the group consisting of insulin resistance disease, hypertension, diabetes, obesity, fatty liver disease, dyslipidemia, liver disease, gout, and hyperuricemia, but is not limited thereto.

As used herein, the term “prevention” refers to any activity that inhibits or delays a metabolic disease by administering the composition of the present invention.

As used herein, the term “treatment” refers to any activity that improves or beneficially changes a symptom of a subject suspected of having or diagnosed with a metabolic disease by administering the composition of the present invention.

In the present invention, the composition may exhibit an effect of improving insulin resistance.

The improvement of insulin resistance may be achieved by the restoration of impaired insulin signaling through normalization of intracellular Ca²⁺, inhibition of lipid accumulation, and rescue of intracellular localization of a PH domain, but is not limited thereto.

The normalization of intracellular Ca²⁺ may be achieved by regulating the expression of essential Ca²⁺ channels or transporters involved in the regulation of intracellular Ca²⁺ concentration, but is not limited thereto.

Additionally, the expression of transporters associated with the normalization of intracellular Ca²⁺ may specifically be downregulating the expression of a store-operated channel (SOC)-related gene, but is not limited thereto.

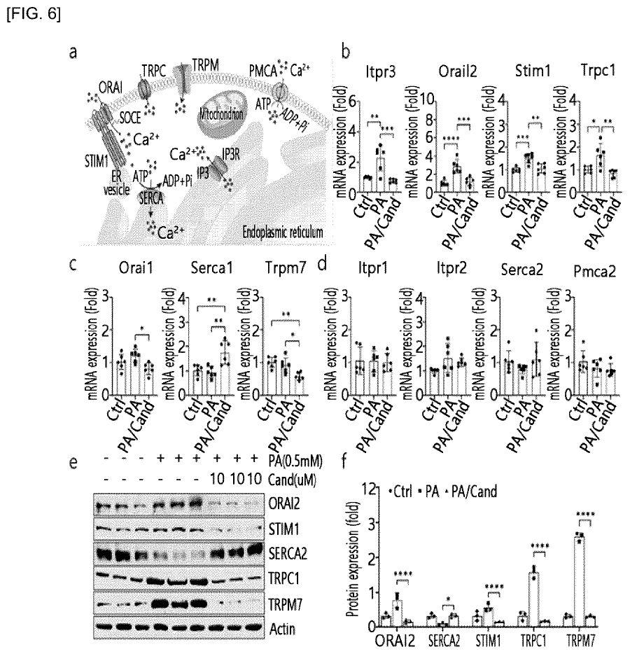

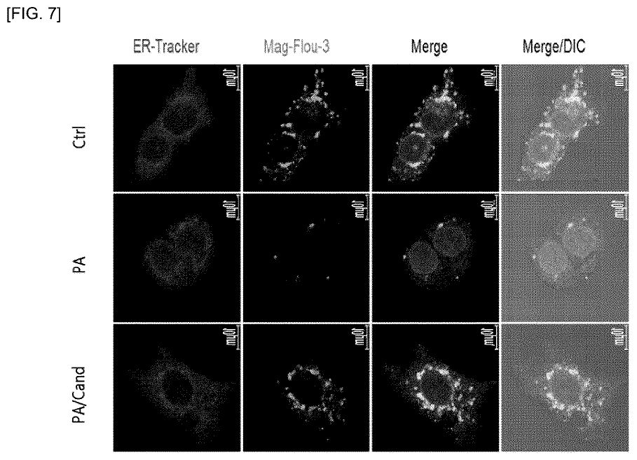

In one embodiment of the present invention, it was confirmed whether candesartan treatment affects the expression of transporters associated with the normalization of intracellular Ca²⁺. As a result, it was confirmed that, by regulating SOC-mediated Ca²⁺ entry into cells, candesartan treatment normalized abnormal intracellular Ca²⁺ and Ca²⁺ homeostasis in the endoplasmic reticulum in palmitic acid-treated HepG2 cells (Examples 3 and 4, and FIGS. 6 and 7).

In the present invention, the SOC-related gene may be inositol 1,4,5-trisphosphate receptor type 3(Itpr3), Orai2, stromal interaction molecule 1(Stim1), and transient receptor potential canonical 1(Trpc1), but is not limited thereto.

Additionally, in the present invention, the composition may exhibit an effect of improving fatty liver.

The effect of improving fatty liver may be achieved by reducing hepatic triglyceride content, ALT, AST, triglycerides, and low-density lipoprotein cholesterol levels, infiltration of inflammatory macrophages, and liver inflammation, but is not limited thereto.

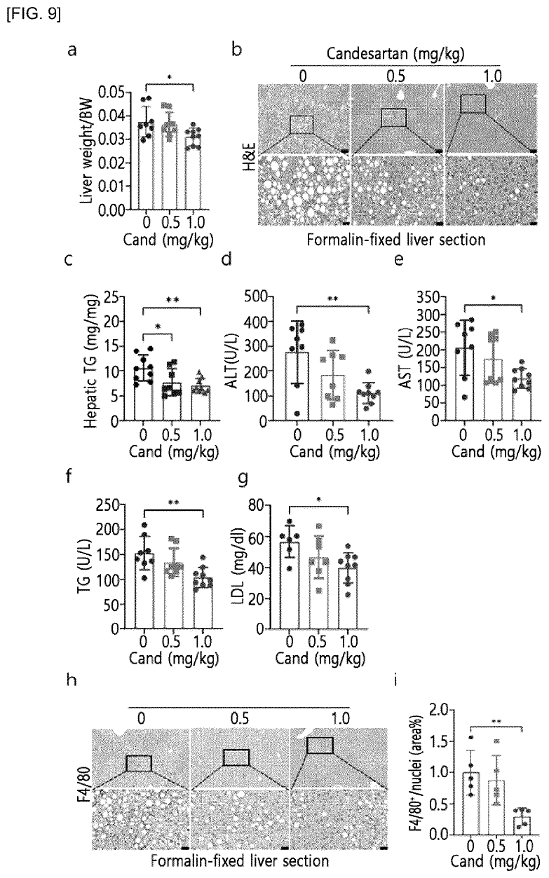

In one embodiment of the present invention, to analyze the effect of improving fatty liver and the blood lipid profile of candesartan, the liver, kidney, and fat tissues of mice were dissected after the experiment, and serum biochemical analysis of ALT, AST, TG, and LDL-cholesterol was performed. As a result, it was confirmed that administration of candesartan improved the biochemical and histological hepatic parameters of mice. In addition, it was confirmed that candesartan significantly reduced serum levels of triglycerides and low-density lipoprotein cholesterol, and decreased the expression of F4/80, a macrophage marker, in subcutaneous and visceral adipose tissue through immunohistochemical staining of F4/80 (Example 6 and FIG. 9).

Additionally, in the present invention, the composition may exhibit an effect of improving kidney function and gout.

The effect of improving kidney function and gout may be achieved by normalizing through a decrease in the levels of serum creatinine (CRE) and uric acid (UA), which are markers of kidney function, but is not limited thereto.

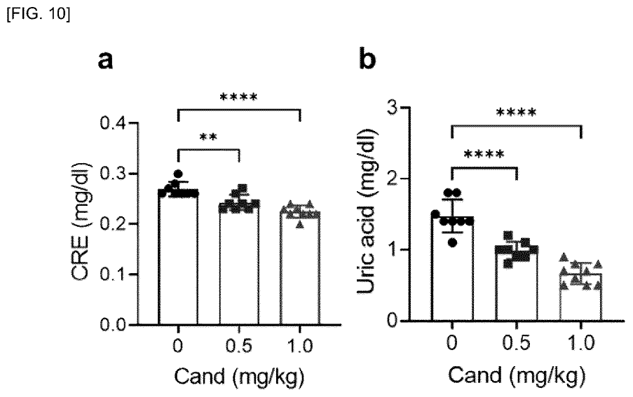

In one embodiment of the present invention, to confirm the effect of candesartan on improving renal function and gout, serum biochemical analysis of creatinine and uric acid was performed. As a result, improvements in biochemical renal and gout parameters were confirmed (Example 7 and FIG. 10).

The pharmaceutical composition of the present invention may contain 0.001 wt % to 80 wt % of candesartan or azilsartan, specifically 0.001 wt % to 70 wt %, and more specifically 0.001 wt % to 60 wt %, based on the total weight of the composition, but is not limited thereto.

In addition, the pharmaceutical composition may further comprise a pharmaceutically acceptable carrier, excipient, or diluent commonly used in the manufacture of pharmaceutical compositions, and the carrier may include non-naturally occurring carriers. The carrier, excipient, or diluent may include lactose, dextrose, sucrose, sorbitol, mannitol, xylitol, erythritol, maltitol, starch, acacia gum, alginate, gelatin, calcium phosphate, calcium silicate, cellulose, methylcellulose, amorphous cellulose, polyvinyl pyrrolidone, water, methyl hydroxybenzoate, propyl hydroxybenzoate, talc, magnesium stearate, and mineral oil.

In addition, the pharmaceutical composition may be used in the form of tablets, pills, powders, granules, capsules, suspensions, oral solutions, emulsions, syrups, sterile aqueous solutions, non-aqueous solvents, suspensions, emulsions, lyophilized preparations, transdermal absorbents, gels, lotions, ointments, creams, patches, cataplasmas, pastes, sprays, skin emulsions, skin suspensions, transdermal delivery patches, drug-containing bandages, or suppositories, respectively, by formulating according to a conventional method. Specifically, during the formulation, the pharmaceutical composition may be prepared by using diluents or excipients, such as fillers, weighting agents, binders, wetting agents, disintegrants, and surfactants that are commonly used. Solid preparations for oral administration include tablets, pills, powders, granules, and capsules, but are not limited thereto. Such solid preparations may be prepared by mixing one or more excipients, including starch, calcium carbonate, sucrose, lactose, and gelatin. Further, in addition to simple excipients, lubricants including magnesium stearate and talc may be used. In addition to oral liquid preparations and liquid paraffin, various excipients such as wetting agents, sweeteners, flavoring agents, and preservatives may be added to the preparation. Preparations for non-oral administration include sterilized aqueous solutions, non-aqueous solvents, suspensions, emulsions, lyophilized preparations, and suppositories. As non-aqueous solvents and suspending agents, propylene glycol, polyethylene glycol, vegetable oils such as olive oil, injectable esters such as ethyl oleate, and the like may be used. As bases for suppositories, Witepsol, Macrogol, Tween 61, cocoa butter, lauric fat, glycerogelatin, and the like may be used.

Another aspect of the present invention for achieving the above objects provides a method for preventing or treating a metabolic disease, comprising administering the pharmaceutical composition to a subject.

As used herein, the terms “pharmaceutical composition”, “metabolic disease”, and “prevention” are as described above.

As used herein, the term “subject” may be any animal, including humans, that has or has developed a metabolic disease of the present invention. An effect of preventing or treating a metabolic disease can be obtained by administering the pharmaceutical composition of the present invention to a subject. Additionally, the subject may include or exclude humans.

The pharmaceutical composition of the present invention is administered in a pharmaceutically effective amount.

As used herein, the term “administration” refers to introducing the pharmaceutical composition of the present invention to a subject by any appropriate method, and the route of administration may be through various oral or parenteral routes capable of reaching the target tissue.

The pharmaceutical composition may be appropriately administered to a subject using a conventional method, route of administration, and dose used in the art according to the purpose or need. Examples of the route of administration include oral, parenteral, subcutaneous, intraperitoneal, intrapulmonary, and intranasal administration. Examples of parenteral administration include intramuscular, intravenous, intraarterial, intraperitoneal, and subcutaneous administration. Additionally, appropriate dose and frequency of administration may be selected according to a known method in the art, and the actual dose and frequency of administration of the pharmaceutical composition of the present invention may be appropriately determined by various factors such as the type of symptom to be treated, route of administration, gender, health conditions, diet, age and weight of the subject, and severity of the disease.

As used herein, the term “pharmaceutically effective amount” refers to an amount that is sufficient to inhibit or alleviate vascular permeability increase at a reasonable benefit-risk ratio applicable to medical use. The effective dosage level may be determined based on factors including the type and severity of the subject, age, gender, drug activity, sensitivity to the drug, administration method, administration time, route of administration and elimination rate, treatment duration, and concurrently used drugs, as well as other well-known factors in the medical field. For example, it may be administered at a dose of 0.01 mg/kg to 500 mg/kg daily, specifically 10 mg/kg to 100 mg/kg, and the administration may be carried out once a day or divided into several times.

The composition of the present invention may be administered as a stand-alone treatment or in combination with other treatments, and may be administered sequentially or simultaneously with conventional treatments. The composition of the present invention may be administered in single or multiple doses. It is important to administer the minimal effective amount that maximizes efficacy without causing side effects, while taking all of the above elements into account, and the amount may be readily determined by a person of ordinary skill in the art.

The composition of the present invention may be used alone to prevent or treat sarcopenia or in combination with methods using surgery, hormone therapy, drug therapy, and biological response modifiers.

Still another object of the present invention for achieving the above objects provides a food composition for preventing or improving a metabolic disease, comprising candesartan or azilsartan.

As used herein, the terms “candesartan”, “azilsartan”, “metabolic disease”, and “prevention” are as described above.

As used herein, the term “improvement” refers to any activity that, by consuming the composition of the present invention, results in at least a reduction in a parameter related to the condition of a metabolic disease, such as the severity of a symptom.

When the composition of the present invention is used as a health functional food additive, candesartan, azilsartan, or a physiologically acceptable salt thereof may be added as is or used in combination with other food products or food ingredients, and may be used appropriately according to a conventional method. The mixing amount of active ingredients may be appropriately determined depending on the intended use (prevention, health, or therapeutic treatment).

The food composition of the present invention can be expected to have an effect of preventing, treating, or improving metabolic diseases as it can be consumed on a daily basis, and therefore it can be very useful for health promotion purposes.

As used herein, the term “food” or “food product” includes meat, sausages, bread, chocolate, candies, snacks, confectionery, pizza, ramen, other noodles, gums, dairy products including ice cream, various soups, beverages, tea, drinks, alcoholic beverages, vitamin complexes, health functional foods, and health foods, and encompasses all foods in the conventional sense.

As used herein, the term “health functional food” is equivalent to the term “food for special health use (FoSHU)”. It refers to a food product that is processed to effectively exhibit bioregulatory functions and possesses high medical and therapeutic effects in addition to providing nutrition. As used herein, the term “functional” refers to having beneficial effects for health purposes, such as regulating nutrients in relation to the structure and function of the human body or promoting physiological actions. The health functional food of the present invention may be prepared using a conventional method in the art, and raw materials and ingredients conventionally added in the art may also be added during the preparation process. In addition, the health functional food may be prepared in any formulation without limitation, as long as the formulation is recognized as food.

The health functional food refers to a food product with a more active health maintenance or promotion effect than general food, and health supplement food refers to a food product for the purpose of health supplementation. In some cases, the terms “health functional food”, “health food”, and “health supplement food” are used interchangeably.

Specifically, the health functional food refers to a food product that provides a specific health benefit when ingested, which is prepared by adding the composition of the present invention to food materials such as beverages, teas, spices, gums, and confectionery, or by manufacturing the same in the form of capsules, powders, suspensions, or the like.

The food composition may further comprise a physiologically acceptable carrier. The type of carrier is not particularly limited, and any carrier may be used as long as it is conventionally used in the art.

Additionally, the food composition may comprise additives conventionally used in food compositions to enhance aroma, taste, appearance, or the like. Examples of the additives include vitamins A, C, D, E, B1, B2, B6, and B12, niacin, biotin, folate, and pantothenic acid. Additionally, the additives may include minerals such as zinc (Zn), iron (Fe), calcium (Ca), chromium (Cr), magnesium (Mg), manganese (Mn), and copper (Cu), as well as amino acids such as lysine, tryptophan, cysteine, and valine.

Additionally, the food composition may comprise food additives such as preservatives (e.g., potassium sorbate, sodium benzoate, salicylic acid, and sodium dehydroacetate), sterilizers (e.g., bleaching powder, high-strength bleaching powder, and sodium hypochlorite), antioxidants (e.g., butylated hydroxyanisole (BHA) and butylated hydroxytoluene (BHT)), coloring agents (e.g., tar dyes), coloring agents (e.g., sodium nitrite and sodium ascorbate), bleaching agents (e.g., sodium sulfite), seasonings (e.g., monosodium glutamate (MSG)), sweeteners (e.g., dulcin, cyclamate, saccharin, and sodium), flavoring agents (e.g., vanillin and lactones), leavening agents (e.g., alum and D-potassium hydrogen tartrate), fortifiers, emulsifiers, thickeners (stabilizers), coating agents, gum bases, anti-foaming agents, solvents, and modifiers. The additives may be selected based on the food type and used in an appropriate amount.

For example, the food composition of the present invention may be used as a health beverage composition, and in such a case, it can contain various flavoring agents or natural carbohydrates as additional ingredients like a regular beverage. The aforementioned term “natural carbohydrates” may include the following: monosaccharides such as glucose and fructose; disaccharides such as maltose and sucrose; polysaccharides such as dextrin and cyclodextrin; and sugar alcohols such as xylitol, sorbitol, and erythritol. Sweeteners may include natural sweeteners, such as thaumatin and stevia extract, and synthetic sweeteners such as saccharin and aspartame. The proportion of the natural carbohydrates may be generally about 0.01 g to 0.04 g, specifically about 0.02 g to 0.03 g, per 100 mL of the health beverage composition of the present invention.

In addition to the above, the health beverage composition may include various nutrients, vitamins, electrolytes, flavoring agents, coloring agents, pectic acid and salts thereof, alginic acid and salts thereof, organic acids, protective colloidal thickeners, pH adjusting agents, stabilizing agents, preservatives, glycerin, alcohols, or carbonation agents. The health beverage composition may also contain fruit pulp for the production of natural fruit juice, fruit juice drinks, or vegetable drinks. These ingredients may be used independently or in combination.

The proportion of these additives is not particularly important, but is typically selected in the range of 0.01 parts to 0.1 parts by weight per 100 parts by weight of the health beverage composition of the present invention.

Still another object of the present invention for achieving the above objects provides a feed composition for preventing or improving a metabolic disease, comprising candesartan or azilsartan.

As used herein, the terms “candesartan”, “azilsartan”, “metabolic disease”, “prevention”, and “improvement” are as described above.

As used herein, the term “feed” refers to any natural or artificial diet, meal, or ingredient of a meal that is intended for or suitable for consumption, ingestion, and digestion by animals.

The type of feed is not particularly limited, and commonly-used feed in the art may be used. Non-limiting examples of the feed include: plant-based feed such as grains, root crops, food processing by-products, algae, fibrous materials, pharmaceutical by-products, fats and oils, starches, gourds, or grain by-products; and animal-based feed such as proteins, minerals, fats and oils, mineral-based substances, single-cell proteins, animal plankton, or food. These examples may be used alone or in combination of two or more.

The candesartan or azilsartan of the present invention included in the feed composition of the present invention may vary depending on the purpose and conditions of use of the feed. For example, it may be included in an amount of 0.01 wt % to 100 wt %, more specifically 1 wt % to 80 wt %, based on the total weight of the livestock feed composition.

Still another object of the present invention for achieving the above objects provides use of a composition comprising candesartan or azilsartan in preventing, improving, or treating a metabolic disease.

Still another object of the present invention for achieving the above objects provides use of candesartan or azilsartan, or a composition comprising the same, for the manufacture of a medicament, food product, or feed for preventing, improving, or treating a metabolic disease.

As used herein, the terms “candesartan”, “azilsartan”, “metabolic disease”, “prevention”, “improvement”, “treatment”, “medicament”, “food”, and “feed” are as described above.

### MODE FOR CARRYING OUT THE INVENTION

Hereinafter, the present invention will be described in detail by way of Examples. The Examples are provided to specifically explain the present invention. The scope of the present invention is not limited by the Examples.

### Experimental Example 1. Animal Models

Male C57BL/6 mice obtained from Orient Bio Inc. (Korea) were used in experiments according to a protocol approved by the Animal Ethics Committee of the Gachon University Lee Gil Ya Cancer and Diabetes Institute (LCDI-2021-0002). After 1 week of adaptation to the control AlN93G diet, 8-week-old male mice were fed a 60% high-fat diet (HFD) for 8 weeks, maintaining a 12-hour light/dark cycle with free access to food and water. After 8 weeks of HFD feeding, mice were assigned to three groups, with four per cage, and were orally administered vehicle or 0.5 mg/kg or 1.0 mg/kg candesartan cilexetil (in PBS) daily for 3 weeks while feeding the same HFD. During the observation period, the body weight and food intake of mice were monitored every 3 days. Food intake was measured for each cage and divided by the number of mice to obtain the total amount of food consumed by each mouse per week.

### Experimental Example 2. Cell Lines and Cell Culture Conditions

Human HepG2 cells were obtained from the American Type Culture Collection (ATCC, Manassas, VA, USA) and confirmed to be mycoplasma-free.

The HepG2 cells were grown in DMEM (Welgene, Korea) containing 10% fetal bovine serum (FBS) and 1× penicillin-streptomycin. CHO-IR/IRS1 cells were grown in F12 medium supplemented with 10% FBS and 1× penicillin-streptomycin. At 70-80% confluency, transient transfection of HepG2 or CHO-IR/IRS1 cells was performed using Lipofectamine 2000 (Invitrogen) containing 1 μg or 10 μg of total DNA, and cells were maintained at 37° C. in 5% CO2.

### Experimental Example 3. Immunoblotting

Tissues and cells were lysed using a modified RIPA buffer (50 mM Tris-HCl pH 7.4, 1% Triton X-100, 150 mM NaCl, 1 mM EDTA; Cell Signaling Technology) freshly supplemented with 1 mM Na3VO4, 10 M NaF, and protease inhibitor cocktail (Complete Mini, Roche). Equal amounts of proteins were separated on 4-12% gels and electrophoretically transferred to polyvinylidene difluoride (PVDF) membranes. Immunoblots were visualized with horseradish peroxidase-conjugated secondary antibodies and enhanced chemiluminescence kits (ECL kits; GE Healthcare Life Sciences) using a charge-coupled device camera (Amersham ImageQuant 800).

### Experimental Example 4. Immunohistochemistry (IHC)

Tissues were fixed in 10% neutral buffered formalin and embedded in paraffin. For liver and fat sections, horseradish peroxidase-conjugated secondary antibodies to F4/80 (anti-rat lgG) containing 3,3′-diamino-benzidine (DAB) tetrahydrochloride substrate (DAKO) were used. All sections were counterstained with hematoxylin (Thermo Scientific). After mounting, the stained slides were captured with a 3DHISTECH scanner (Budapest, Hungary), and DAB was quantified using NIH ImageJ software (https://imagej.nih.gov/ij/, 1997-2017).

### Experimental Example 5. qRT-PCR Analysis

Total RNA was extracted from HepG2 cells and frozen liver, kidney, and adipose tissues using TRIzol (Invitrogen) according to the manufacturer's protocol. RNA concentrations were measured on a NanoDrop spectrophotometer (Thermo Scientific), and cDNA synthesis and real-time RT-PCR were performed using the TB Green Premix Ex Taq cDNA Synthesis Kit (TaKaRa Bio, Tokyo, Japan) with 1 μg of total RNA and random hexamers, on a BioRad CFX384 system. All expression values were normalized to cyclophilin A mRNA levels. The primer sequences used are as shown below (Tables 1 and 2).

TABLE 1

| Gene | Gene Bank No. | Primer | Primer Sequence |
|---|---|---|---|
| ITPR3 | NM_080553.3 | Forward | 5′-CGTCTGGGCTTTGTGGATGTG |
| | | Reverse | 5′-TGACCTGAAGGAAGGGCAGTG |
| ORAI1 | NM_175423.3 | Forward | 5′-GGAAATGGCTCGGGGACAAA |
| | | Reverse | 5′-CTAGCCCCAGCAACACAGTT |
| ORAI2 | NM_178751.3 | Forward | 5′-ACACATCCCAAGAGCCACAG |
| | | Reverse | 5′-ACCCTTATTCATGCGGGGAC |
| PMCA2 | NM_009723.6 | Forward | 5′-CTA GAG CCG GCA AGA CAT GG |
| | | Reverse | 5′-CTG GCA TCT ACA TGG TCG GG |
| SERCA1 | NM_007504.2 | Forward | 5′-TAAAGTCCTCCACCCTCGTC |
| | | Reverse | 5′-CTAGTCAGTTGCCTTGTGCC |
| SERCA2 | NM_001110140.3 | Forward | 5′-GCTTCTTCTGTAGGTCTGATGGT |
| | | Reverse | 5′-TAGGCGAAGGGACAGAAACC |
| STIM1 | NM_009287.4 | Forward | 5′-CCCATCAGCATCAGCACTAC |
| | | Reverse | 5′-TGGTTCAAATATAACATCCTTGCC |
| TRPC1 | NM_011643.3 | Forward | 5′-GCAAACCCGTTTTGTTCGCA |
| | | Reverse | 5′-CACTGATAGCTGCCCCTCTT |
| TRPM4 | NM_175130.4 | Forward | 5′-GAAGCCCGGGATCGGATTAG |
| | | Reverse | 5′-TCAGCAACTCCTTTCGGGTC |
| TRPM7 | NM_021450.2 | Forward | 5′-GCA GCT TTG TTA CCG GAT TGG |
| | | Reverse | 5′-ACG GGC TTA AAT GGA GAA GCA |
| CCI2 | NM_011331 | Forward | 5′-TTAAAAACCTGGATCGGAACCAA |
| | | Reverse | 5′-GCATTAGCTTCAGATTTACGGG |
| F4/80 | NM_153100.1 | Forward | 5′-GCCTATTATCTATACCCTCCAGCACATC |
| | | Reverse | 5′-TCCATCTCCCATCCTCCACATCAG |
| IL1B | NM_008361.4 | Forward | 5′-TCCATTGAGGTGGAGAGCTTTC |
| | | Reverse | 5′-CTACAGGCTCCGAGATGAACAAC |
| TNF-a | NM_013693 | Forward | 5′-AGACCCTCACACTCAGATCATCTTC |
| | | Reverse | 5′-CCTCCACTTGGTGGTTTGCT |
| ATF3 | NM_007498.3 | Forward | 5′-CCTTATCATCCCGGCCAGTC |
| | | Reverse | 5′-GTCCCACCCCACCTATCAAG |
| ATF4 | NM_009716.3 | Forward | 5′-CGCTCTTCACGAAATCCAGC |
| | | Reverse | 5′-TCATGTTGTGGGGCTTTGCT |
| ATF6 | NM_001081304.1 | Forward | 5′-CTTGCCTTGTACGCAACTCC |
| | | Reverse | 5′-GCACTGCCACCAGAGAATTG |
| IRE1a | NM_023913.2 | Forward | 5′-GCCCTCAGCATGATGGACTT |
| | | Reverse | 5′-TGTCTTGCCTCCAAAGGATGT |
| XBP1 | NM_013842.3 | Forward | 5′-ATCGCAGGGAGGGTCATTTG |
| | | Reverse | 5′-TGGGGTCAAGAGGGTCAGAA |
| XBP1s | | Forward | 5′-GAGTCCGCAGCAGGTG |
| | | Reverse | 5′-GTGTCAGAGTCCATGGGA |
| CEBPB | NM_009883.4 | Forward | 5′-GACAAGCTGAGCGACGAGTA |
| | | Reverse | 5′-GTCAGCTCCAGCACCTTGT |
| ChREBP | NM_021455.4 | Forward | 5′-ATGCGGGACATGTTTGATGA |
| | | Reverse | 5′-TCGGATGAGGATGCTGAACAC |
| NFKB | NM_008689 | Forward | 5′-TGGCAGCTCTTCTCAAAGCA |
| | | Reverse | 5′-CCCAAGAGTCGTCCAGGTCAT |
| NRF2 | NM_010902.5 | Forward | 5′-GCCCTCAGCATGATGGACTT |
| | | Reverse | 5′-TGTCTTGCCTCCAAAGGATGT |
| Nurr1 | NM_013613.2 | Forward | 5′-AGCTGGTTGAAGTGTCCAGT |
| | | Reverse | 5′-GCAGGTTAGGAAATAGCGACA |
| Nur77 | NM_010444.2 | Forward | 5′-GGTTCCCTGGACGTTATCCG |
| | | Reverse | 5′-GGCGGAGGATGAAGAGTTCC |
| PPARa | NM_011144.6 | Forward | 5′-CGCTCCCGACCCATCTTTAG |
| | | Reverse | 5′-ACTGGGCTACATCCTCGACT |
| PPARd | NM_011145.3 | Forward | 5′-ACCAGCAGCCTAAAAGCAGT |
| | | Reverse | 5′-CCCAGAGAGAGCGTAGTGTT |
| PPARg | NM_011146 | Forward | 5′-CAGGCCGAGAAGGAGAAGCT |
| | | Reverse | 5′-GGCTCGCAGATCAGCAGACT |
| SREBP1c | \|NM_011480.3 | Forward | 5′-GTACCTGCGGGACAGCTTAG |
| | | Reverse | 5′-TCAGGTCATGTTGGAAACCA |
| G6PC | NM_008061.3 | Forward | 5′-GGCGCAGCAGGTGTATACTATGT |
| | | Reverse | 5′-CAAACACCGGAATCCATACGT |
| PEPCK | NM_011044.2 | Forward | 5′-CGCAAGCTGAAGAAATATGACAA |
| | | Reverse | 5′-TCGATCCTGGCCACATCTC |
| FABP1 | NM_017399.5 | Forward | 5′-AAGGGAAGGACATCAAGGGG |
| | | Reverse | 5′-GGTGAACTCATTGCGGACCA |
| FABP4 | NM_024406.4 | Forward | 5′-GTCACCATCCGGTCAGAGAG |
| | | Reverse | 5′-CCTGTCGTCTGCGGTGATTT |
| FABP5 | NM_010634.3 | Forward | 5′-ACGGGAAGGAGAGCACGATA |
| | | Reverse | 5′-AGCCCTCATTGCACCTTCTC |
| CD36 | NM_007643 | Forward | 5′-ATACAGAGTTCGTTATCTAGCCAAGGA |
| | | Reverse | 5′-CCATTGGGCTGTACAAAAGACA |
| Ldlr | NM_001252658.1 | Forward | 5′-CCCTTCTCCTTGGCCATCTAT |
| | | Reverse | 5′-GAGTCGATTGGCACTGAAAATG |
| Cidea | NM_007702.2 | Forward | 5′-GGGACAACACGCATTTCATG |
| | | Reverse | 5′-GTTGCTTGCAGACTGGGACAT |
| Cideb | NM_026925.3 | Forward | 5′-ACAGTCCGGAAAGGACTGACA |
| | | Reverse | 5′-CACTCCACGTAGCAGCAAGGT |
| Cidec | NM_178373.3 | Forward | 5′-CCATCAGAACAGCGCAAGAA |
| | | Reverse | 5′-ACTGGGCTACATCCTCGACT |
| Acc1 | NM_133360.2 | Forward | 5′-CTGAAGCAGATCCGCAGCTT |
| | | Reverse | 5′-GGTGAGATGTGCTGGGTCATG |
| Acc2 | NM_133904.2 | Forward | 5′-TGGAGTCCATCTTCCTGTCC |
| | | Reverse | 5′-CGAATATGGTCGTTTCTGAAAGT |
| FAS | NM_007988.3 | Forward | 5′-GAAGAAGTGTCTGGACTGTGTCATTT |
| | | Reverse | 5′-TTAATTGTGGGATCAGGAGAGCAT |
| Cpt1 | NM_013495.2 | Forward | 5′-TCGTGGTGGTGGGTGTGAT |
| | | Reverse | 5′-GGGTCCGATTGATCTTTGCA |

TABLE 2

| Gene | Gene Bank No. | Primer | Primer Sequence |
|---|---|---|---|
| hITPR1 | NM 001099952.2 | Forward | 5′_CGG AGC AGG GTA TTG GAA CA |
| | | Reverse | 5′_CTG AGG GCT GAA ACT CCA GG |
| hITPR2 | NM_002223.3 | Forward | 5′_CTT CAT CAG CAC CTT GGG GT |
| | | Reverse | 5′_GGC ACA CCT TGA AAA GGC AG |
| hITPR3 | NM_002224.3 | Forward | 5′_GCT TCA TCA GCA CTT TGG GG |
| | | Reverse | 5′_ATG GGG CAC ACC TTG AAG AG |
| hORAI1 | NM_032790.3 | Forward | 5′_AAG CTT AAA GCC TCC AGC CG |
| | | Reverse | 5′ GTG GGT AGT CGT GGT CAG C |
| hORAI2 | NM_001271819.1 | Forward | 5′_ATG GGA TGG AGA GCC TGA GT |
| | | Reverse | 5′_GGT ACT GGT ACT GCG TCT CC |
| hPMCA2 | NM_001001331.3 | Forward | 5′_GGT GTG AAG AAG GGG GAT GG |
| | | Reverse | 5′_CTA GGC TGG CAT TCG CAC TA |
| hSERCA1 | NM_004320.4 | Forward | 5′ GTG GGA GCT GGT GAT AGA GC |
| | | Reverse | 5′_TCA CCT TCC TCA AAC CAG GC |
| hSERCA2 | NM_001681.3 | Forward | 5′_TTG CTC GAG TTG AAC CCT CC |
| | | Reverse | 5′_CGG CTT TCT TCA GAG CAG GA |
| hSTIM1 | NM_003156.3 | Forward | 5′ TTG TGT CTC CCT TGT CCA TGC |
| | | Reverse | 5′ GGG TCA AAT CCC TCT GAG ATC C |
| hTRPC1 | NM_001251845.1 | Forward | 5′_AAG CTT TTC TTG CTG GCG TG |
| | | Reverse | 5′ CTC CCA AGC ACA TCT ACG CA |
| hTRPM7 | NM_017672.5 | Forward | 5′_GAG GAG GAG GAG GTG GAG TT |
| | | Reverse | 5′_TCC CTC TTG GTC AAA GTG CT |

### Experimental Example 6. Gene Set Enrichment Analysis (GSEA)

GSEA was performed on expressed genes according to the software manual. Gene sets with a nominal p value <0.05 and an FDR <0.25 were considered significant, and the exact p values and FDR q values used in the GSEA analysis are provided in the Source Data. Clustered heatmap visualization was performed using Java TreeView (v.4.0).

### Experimental Example 7. RNA Sequencing and Data Analysis

Total RNA was isolated from liver tissues using RNAiso Plus reagent (Takara, Shiga, Japan) and treated with RNase-free DNase (Roche, Mannheim, Germany) to isolate genomic DNA. The RNA concentration and integrity of each sample were measured using a 2100 Bioanalyzer (Agilent) with an RNA integrity threshold of ≥8. A cDNA library was prepared using the TruSeq RNA Library Kit (Illumina, San Diego, CA, USA). The generated library was amplified by PCR for 15 cycles and then sequenced on the HiSeq™ 2000 sequencing system (Illumina) at Macrogen (Seoul, Korea) to generate 100 bp paired-end reads. Differentially expressed genes were analyzed using Fisher's exact test, and the resulting p values were subjected to multiplicity correction using the Benjamini-Hochberg method to control the false discovery rate (FDR). Differentially regulated genes with an FDR value less than 0.05 were considered significant, and expressed genes were defined as having detectable expression in at least half of all samples. The expressed genes were subjected to principal component analysis (PCA). Additionally, the enriched signaling pathways were analyzed based on the identified differentially expressed genes using the Ingenuity Pathway Analysis software (IPA, QIAGEN). For the analysis, gene expression data meeting any of the following criteria were excluded from all samples: (a) log 2-fold-change (log 2FC) value between −1 and 1; (b) p>0.05; (c) false discovery rate (FDR) >0.05; and (d) FPKM <1.

### Experimental Example 8. Statistical Analysis

The sample size for all experiments was based on preliminary results and previous experience conducting related experiments. Power calculations were not used to determine the sample size. Animals in each experimental group were randomly assigned, and no animals were excluded from the statistical analysis. Unless otherwise stated, all data are presented as mean±standard deviation. Statistical comparisons between groups were performed using an unpaired Student's t-test or one-way ANOVA, together with Tukey's post hoc test or Holm-Sidak's post hoc test for multiple comparisons. All data were subjected to D'Agostino and Pearson (n>8) or Shapiro-Wilk (n<8) normality test prior to analysis. Groups that failed the normality test (p<0.05) underwent an outlier test (ROUT, Q=1%) to determine whether the outliers were responsible for the failure of the normality test. Data were excluded from the group if they passed the normality test by excluding the outliers, and data were not excluded if the statistical results did not change. The Kruskal-Wallis test was used for nonparametric statistical analysis of data that were not normally distributed. Data analysis and graph creation were performed using GraphPad Prism 9.4 (GraphPad Software Inc, San Diego, CA).

### Example 1. Analysis of Angiotensin-II-Receptor Blockers (ARBs) that Alleviate Insulin Resistance

Certain ARBs can attenuate palmitic acid (PA)-induced insulin resistance in HepG2 cells by reducing lipid accumulation and intracellular Ca²⁺ overload. Additionally, excessive intracellular Ca²⁺ inhibits insulin signaling by inhibiting the interaction between the PH domain and membrane-bound PI(3, 4)P₃ or PI(3, 4, 5)P₃, which eliminates the membrane localization of the PH domain and leads to impaired insulin signaling.

### Example 1-1. Restoration of Impaired Insulin Signaling

Herein, it was evaluated whether, and by what mechanism, ARBs improve insulin resistance by attenuating intracellular Ca²⁺ overload and thereby restoring impaired insulin signaling.

Specifically, nine FDA-approved ARBs (losartan, eprosartan, fimasartan, irbesartan, olmesartan, telmisartan, valsartan, azilsartan, and candesartan) commonly prescribed for the treatment of hypertension in human patients were administered to HepG2 cells at various concentrations (5 μM, 10 μM, and 20 μM) in combination with 0.5 mM palmitic acid for 16 hours. Then, the insulin-stimulated phosphorylation of the key insulin signaling molecule Akt and its downstream signaling molecules GSK3β and FOXO3 was investigated.

As a result, as shown in FIG. 1, palmitic acid treatment significantly attenuated the insulin-stimulated phosphorylation of Akt, GSK3B, and FOXO3 in HepG2 cells, confirming that palmitic acid treatment leads to impaired insulin signaling. Subsequently, it was confirmed that azilsartan and candesartan completely restored the insulin-stimulated phosphorylation of Akt, GSK3B, and FOXO3 in palmitic acid-treated HepG2 cells in a dose-dependent manner. In contrast, the other ARBs did not demonstrate such a recovery effect.

### Example 1-2. Regulation of Intracellular Ca²⁺ Levels and Lipid Accumulation

Subsequently, to determine whether ARBs can regulate intracellular Ca²⁺ levels and lipid accumulation in HepG2 cells treated with palmitic acid, the effects of ARBs were evaluated using confocal microscopy and the fluorescent dyes Calbryte 630 AM (for intracellular Ca²⁺) and BODIPY (for lipids).

Specifically, for intracellular Ca²⁺ concentration and lipid accumulation, HepG2 cells were plated on slide glasses, treated with 0.5 mM palmitic acid for 16 hours, and incubated with 4 μM Fura-2-AM (Teflabs), 5 mM Calbryte 630 AM, or 2 mM BODIPY in PBS for 30 minutes at 37° C. in the dark. The cells were then washed with PBS for 10 minutes. Subsequently, the cells were counterstained with 4,6-diamidino-2-phenylindole (DAPI) to visualize nuclei. After mounting, the stained slides were imaged using a Zeiss LSM 700 or Zeiss LSM 980 laser scanning confocal microscope (Carl Zeiss) and analyzed using ZEN 2010 Software (Carl Zeiss). Intracellular Ca²⁺ was quantified from the microscopic images using the NIH ImageJ software (https://imagej.nih.gov/ij/, 1997-2017).

As a result, as shown in FIG. 2, azilsartan and candesartan significantly reduced the fluorescence intensity for intracellular Ca²⁺ (FIGS. 2a and 2b) and lipid accumulation (FIGS. 2a and 2c) in HepG2 cells. Meanwhile, eprosartan, fimasartan, irbesartan, losartan, olmesartan, and telmisartan did not significantly affect the intracellular Ca²⁺ concentration or lipid accumulation in HepG2 cells (FIGS. 2a to 2c).

These results suggest that azilsartan and candesartan can attenuate lipid accumulation and intracellular Ca²⁺ overload, thereby restoring insulin signaling impaired by palmitic acid treatment.

### Example 1-3. Analysis of Insulin-Stimulated Membrane Localization of the PH Domain of AKT

One mechanism of insulin resistance is that, in the state of obesity and diabetes, an increase in the concentration of calcium in the cytoplasm leads to calcium binding to PI(3, 4, 5)P₃ present in the cell membrane, generating Ca²⁺-PI(3, 4, 5)P₃, which inhibits binding to the PH domain of AKT and suppresses AKT protein activity, thereby inducing insulin resistance. Accordingly, it was evaluated whether azilsartan and candesartan, which were shown in the above examples to attenuate palmitic acid-induced intracellular Ca²⁺ overload, regulate the intracellular localization of the AKT PH domain, which functions as a biosensor for PI(3, 4, 5)P₃.

Specifically, CHO cells or HepG2 cells were transfected with cDNA encoding hAKT PHD (residues 1 to 144) using Lipofectamine (Life Technologies). To monitor the intracellular localization of endogenous AKT, liver sections were antigen activated with 10 mM Tris/HCl (pH 9.0) for 20 minutes, treated with serum-free protein blocking solution (DAKO) for 30 minutes, and permeabilized with 0.5% Tween 20 for 15 minutes. The liver sections were then incubated with anti-AKT PHD antibody (SKB1, #05-591, Sigma-Aldrich) and FITC-conjugated secondary antibody. After mounting, the liver sections were imaged with an LSM 980 laser scanning confocal microscope (Carl Zeiss), and the images were analyzed using ZEN software (Carl Zeiss).

As a result, as shown in FIG. 3, AKT PH mCherry was immediately translocated from the cytoplasm to the plasma membrane after treating cells with 100 nM insulin for 15 minutes, confirming that palmitic acid treatment inhibits the insulin-stimulated membrane localization of the AKT PH domain. When administering azilsartan or candesartan to cells, the impairment induced by palmitic acid was recovered. In contrast, treatment with eprosartan, fimasartan, irbesartan, losartan, olmesartan, and telmisartan did not rescue the impairment induced by palmitic acid (FIG. 3a).

Similarly, in phorbol myristate acetate (PMA)-treated Chinese hamster ovary-insulin receptor (CHO-IR) cells, it was confirmed that insulin-stimulated membrane localization of the AKT PH domain was rescued by treatment with azilsartan or candesartan (FIG. 3b).

These results indicate that attenuation of intracellular Ca²⁺ overload by azilsartan or candesartan may restore palmitic acid-induced impairment of insulin-stimulated membrane localization of the AKT PH domain.

### Example 2. Effect of Candesartan on Improving Insulin Resistance

### Example 2-1. Intracellular Ca²⁺ Homeostasis, Lipid Accumulation, and Insulin Signaling

The results of Example 1 indicate that azilsartan and candesartan have beneficial off-target effects on insulin signaling and intracellular Ca²⁺ homeostasis that are distinct from their known effects as angiotensin receptor blockers. In particular, since candesartan exhibited consistent effects on insulin sensitivity in a dose-dependent manner compared to azilsartan, the effects of candesartan on intracellular Ca²⁺ homeostasis and lipid accumulation were verified in the same manner using confocal microscopy with the fluorescent dyes BODIPY (for lipids) and Fluo-3AM (for intracellular Ca²⁺). Additionally, in order to verify enhanced insulin signaling, immunoblot analysis for AKT substrate of 160 (AS160) was performed using an anti-phospho-AS160 antibody to test whether candesartan has a beneficial effect on glucose uptake.

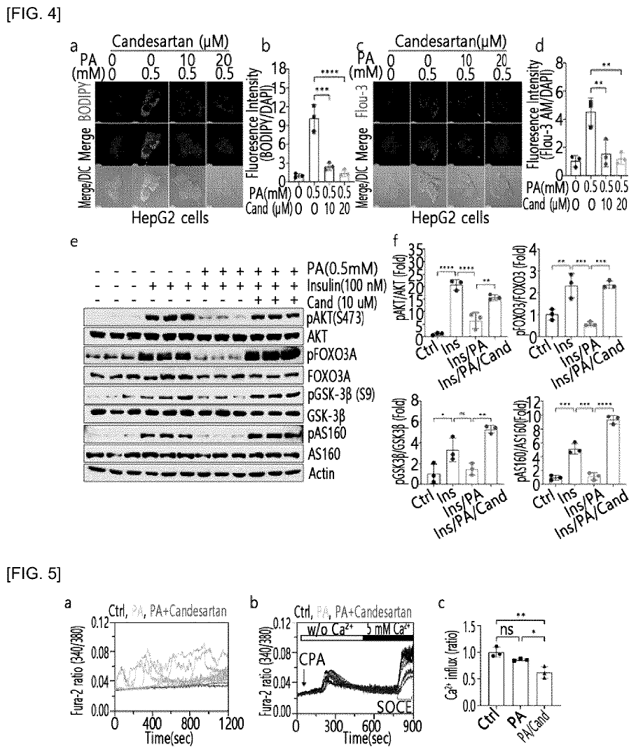

As a result, as shown in FIG. 4, it was confirmed that candesartan significantly reduced palmitic acid-induced lipid accumulation in HepG2 cells in a dose-dependent manner and attenuated intracellular Ca²⁺ overload (FIGS. 4a to 4d).

Furthermore, it was found that candesartan treatment increased the insulin-stimulated phosphorylation of AKT, FOXO3, GSK3B, and AS160 in palmitic acid-treated HepG2 cells compared to control cells (FIGS. 4e and 4f).

These results indicate that candesartan completely restored the impaired insulin signaling by palmitic acid treatment in HepG2 cells.

### Example 2-2. Analysis of Method for Inhibiting Intracellular Ca²⁺ Overload

Based on the results of the effect of candesartan on inhibiting intracellular Ca²⁺ overload, [Ca2+]i signaling was measured using fura-2 AM, a fluorescent dye for cytosolic Ca²⁺, in HepG2 cells 24 hours after palmitic acid treatment.

Specifically, the cells were attached to coverslips and incubated with 4 μM Fura-2 AM and 0.05% Pluronic acid for 15 minutes at room temperature in the dark. The dye-loaded cells were then placed on an inverted microscope and perfused with physiological salt solution to detect [Ca²⁺]i. [Ca2+]i was determined based on the Fura-2 ratio at dual excitation wavelengths of 340 nm and 380 nm (emission wavelength of 530 nm). The emitted fluorescence was monitored with a CCD camera (Q-imaging) attached to an inverted microscope (Nikon, Japan) and analyzed with the Metafluor system (Molecular Devices, USA). For Ca²⁺ influx measurements, the cells were stimulated with the sarco/endoplasmic reticulum Ca²⁺ ATPase inhibitor cyclopiazonic acid (CPA) in a Ca²⁺-free solution to deplete Ca²⁺ stores, and were subsequently treated with PBS containing 5 mM Ca²⁺.

As a result, as shown in FIG. 5, palmitic acid treatment led to persistent intracellular Ca²⁺ overload in HepG2 cells, which was significantly attenuated by candesartan treatment (FIG. 5a). Additionally, the role of candesartan in Ca²⁺ influx into HepG2 cells via SOCs after the depletion of cellular Ca²⁺ stores by cyclopiazonic acid (CPA), a sarcoendoplasmic Ca²⁺-ATPase (SERCA) inhibitor, was determined (FIG. 5b). That is, it was found that candesartan treatment effectively reduced SOC-mediated Ca²⁺ influx into palmitic acid-treated HepG2 cells (FIG. 5c).

### Example 3. Analysis of Essential Ca²⁺ Channel and Transporter Expression Levels

In order to evaluate the method by which candesartan regulates intracellular Ca²⁺ levels in palmitic acid-treated HepG2 cells, qRT-PCR and immunoblot analyses were performed to analyze the expression levels of essential Ca²⁺ channels and transporters in the endoplasmic reticulum.

As a result, as shown in FIG. 6, palmitic acid treatment significantly up-regulated the expression levels of inositol 1,4,5-trisphosphate receptor type 3(Itpr3), Orai2, stromal interaction molecule 1(Stim1), and transient receptor potential canonical 1(Trpc1) compared to the control cells. In contrast, candesartan treatment dramatically down-regulated the expression of SOC-related genes such as Itpr3, Orai2, Stim1, and Trpc1 (FIG. 6b). This result was consistent with the results of Example 3-2 above that candesartan inhibited SOC-mediated Ca²⁺ influx in palmitic acid-treated HepG2 cells. In addition, candesartan upregulated the expression of Serca1, which encodes an essential Ca²⁺ channel or transporter involved in regulating intracellular Ca²⁺ concentration, and downregulated the expression of Orai1 and Trpm7 (FIG. 6c). In contrast, palmitic acid treatment did not change the expression levels of Ca²⁺ channels or transporters, such as Itpr1, Itpr2, Serca2, and plasma membrane calcium transport ATPase 2 (Pmca2), compared to the control cells or cells treated with both candesartan and palmitic acid (FIG. 6d).

Moreover, immunoblot analysis confirmed that candesartan significantly increased the expression of SERCA2 in palmitic acid-treated HepG2 cells, while normalizing the expression of SOC proteins such as ORAI2, STIM1, TRPC1, and TRPM7 (FIGS. 6e and 6f).

### Example 4. Effect of Improving Endoplasmic Reticulum Ca²⁺ Content and Endoplasmic Reticulum Function

To evaluate how candesartan modulates endoplasmic reticulum Ca²⁺ levels in palmitate-treated HepG2 cells, the cells were stained for 2 hours with ER-Tracker Red (BODIPY TR Glibenclamide, ThermoFisher), an ER marker, followed by staining with Mag-Fluo-4, AM Green (ThermoFisher), which specifically binds to endoplasmic reticulum Ca²⁺, and then analyzed.

As a result, as shown in FIG. 7, it was confirmed that palmitic acid treatment decreased the Ca²⁺ content in the endoplasmic reticulum compared to the control cells, whereas candesartan normalized the Ca²⁺ content in the endoplasmic reticulum of palmitic acid-treated HepG2 cells to the same level as the control cells.

These in vitro study results suggest that candesartan treatment normalized the abnormal intracellular Ca²⁺ homeostasis and Ca²⁺ in the endoplasmic reticulum in PA-treated HepG2 cells by regulating SOC-mediated Ca²⁺ entry into cells.

### Example 5. Effect of Candesartan on Improving Insulin Resistance in High-Fat Diet-Fed Mice

To evaluate the therapeutic potential of candesartan-mediated enhancement of insulin signaling, as demonstrated in the in vitro experimental results of Examples 1 to 4 above, the effect of candesartan administration on insulin resistance was investigated in mice fed a high-fat diet (HFD).

For animal in vivo experiments, candesartan cilexetil was used.

Specifically, 8-week-old male C57BL/6 mice were fed an HFD for 8 weeks, followed by daily administration of candesartan at 0.5 mg/kg or 1.0 mg/kg for 21 days while maintaining the same HFD (FIG. 8a). Intraperitoneal glucose tolerance test (IP-GTT) was performed by intraperitoneal injection of glucose at a dose of 1 g/kg body weight after 16 hours of fasting. Blood samples were collected from a small incision at the tip of the tail at 0, 15, 30, 60, and 90 minutes after glucose injection, and blood glucose levels were measured using a glucometer (Accu-Chek Aviva, Roche).

An intraperitoneal insulin tolerance test (IP-ITT) was performed by intraperitoneal injection of insulin at a dose of 0.75 U/kg body weight (Actrapid Penfill, Novo Nordisk) after 4 hours of fasting. Blood glucose levels were measured at 0, 15, 30, 60, and 120 minutes after insulin injection. Homeostasis model assessment for insulin resistance (HOMA-IR) was calculated using the following formula:

(fasting insulin×fasting glucose)/405

As a result, as shown in FIG. 8, mice administered with candesartan showed a gradual dose-dependent decrease in body weight without changes in food intake compared to the control mouse group (FIG. 8b). IP-GTT and IP-ITT showed that mice administered with candesartan had significantly improved glucose tolerance and insulin sensitivity, respectively, as evidenced by a decrease in the area under the curve (AUC) compared to the control group (FIGS. 8c and 8d).

### Example 6. Analysis of the Effect of Candesartan on Fatty Liver Improvement and Blood Lipid Profile

After the experiment, the body composition of mice was analyzed using nuclear magnetic resonance imaging (Minispec Live Mice Analyzer (LF50), Bruker). Frozen liver tissues were homogenized in modified RIPA buffer. Triglyceride content and total protein of the liver lysates were measured using the AU-480 Biochemistry Analyzer, and triglyceride content was normalized to total protein concentration. Additionally, after the completion of the experiment, the mice were fasted for 16 hours, then fed an HFD, followed by isoflurane administration and collection of blood from the abdominal aorta. Liver, kidney, and fat tissues were dissected and stored at −80° C.

Serum biochemical analyses of ALT, AST, TG, and LDL-cholesterol were performed using the AU-480 analyzer (Beckman Coulter, Fullerton, CA, USA).

As a result, as shown in FIG. 9, administration of candesartan decreased the liver weight-to-body weight ratio (FIG. 9a) and improved biochemical and histological liver parameters. These improvements were confirmed by H&E staining of liver sections (FIG. 9b), quantification of hepatic triglyceride content (FIG. 9c), and measurement of serum liver function markers alanine transaminase (ALT) (FIG. 9d) and aspartate transaminase (AST) (FIG. 9e). Additionally, candesartan was found to significantly reduce serum levels of triglycerides and low-density lipoprotein cholesterol (FIGS. 9f and 9g).

Furthermore, immunohistochemical staining of the macrophage marker F4/80 showed that administration of candesartan significantly reduced inflammatory macrophage infiltration and liver inflammation compared to the HFD-fed control group (FIGS. 9h and 9i). In addition, qRT-PCR analysis in adipose tissue revealed that candesartan reduced the expression of F4/80 in both subcutaneous and visceral adipose tissue (FIGS. 9j and 9k).

### Example 7. Effect of Candesartan on Improving Renal Function and Gout

Serum biochemical analyses of creatinine (CRE) and uric acid (UA) were performed using the AU-480 analyzer (Beckman Coulter, Fullerton, CA, USA).

As a result, as shown in FIG. 10, administration of candesartan improved biochemical parameters related to renal function and gout, as confirmed by the measurements of serum renal function markers, creatinine (FIG. 10a) and uric acid (FIG. 10b).

### Example 8. Candesartan-Induced Changes in Hepatic Metabolic Transcriptome

To determine the molecular mechanisms underlying the enhanced metabolic profile observed in mice administered with candesartan, RNA-sequencing was performed on liver tissues from the control group and the candesartan-administered group.

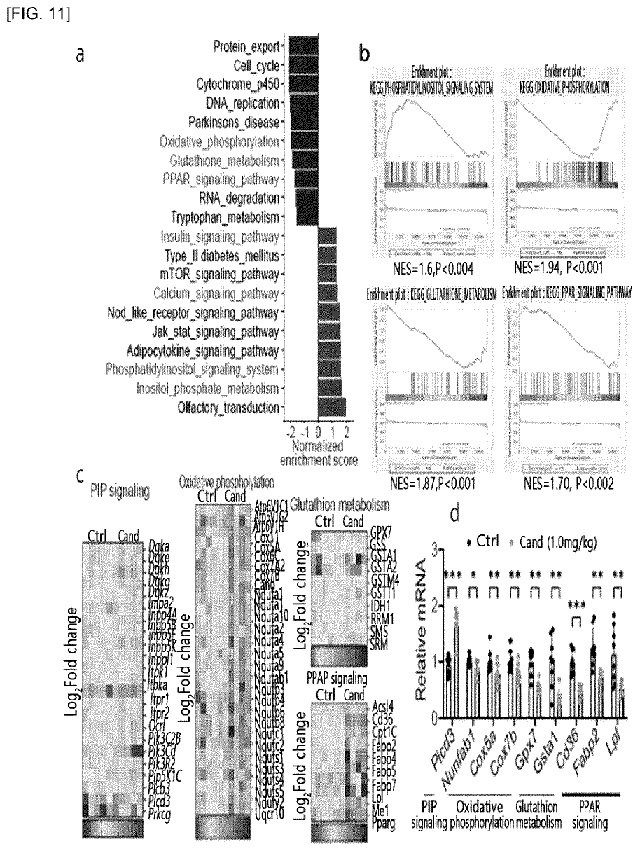

As a result, as shown in FIG. 11, gene set enrichment analysis (GSEA) showed that the oxidative phosphorylation signature and PPAR signaling signature were significantly down-regulated, whereas the PIP signaling system signature and calcium signaling signature were significantly up-regulated in the candesartan-administered group compared to the control group (FIGS. 11a and 11b). Additionally, through the heatmap plots of PIP signaling, oxidative phosphorylation, PPAR signaling, and glutathione metabolism pathways, differential expression in the candesartan-administered group compared to the control group was confirmed (FIG. 11c). Furthermore, using qRT-PCR, dysregulated expression of selected genes associated with each of these pathways was confirmed (FIG. 11d).

In sum, these analysis results confirmed the marked effects of candesartan treatment on gene expression and signaling pathways associated with insulin signaling in HFD-fed mice.

### Example 9. Analysis of SOCE-Related Genes and Protein Expression of Candesartan

To investigate whether candesartan differentially regulates genes associated with hepatic Ca²⁺ homeostasis in HFD-fed mice, hepatic expression levels of essential Ca²⁺ channel and transporter genes were analyzed.

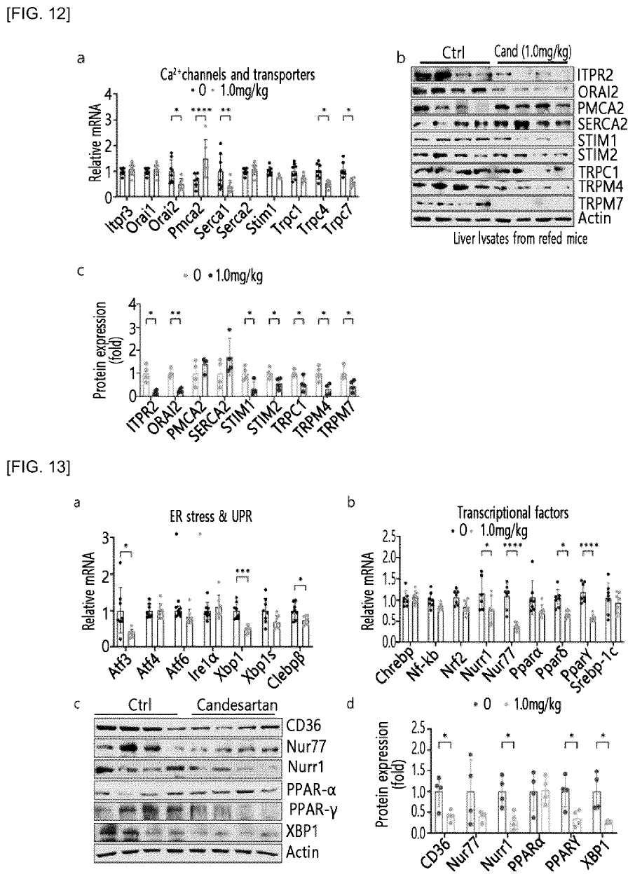

As shown in FIG. 12, the results were consistent with the in vitro results. Administration of candesartan to HFD-fed mice dramatically down-regulated the expression of transporter genes, including Orai2, Serca1, Trpm4, and Trpm7, which are known to increase the expression of Ca²⁺ channels and intracellular Ca²⁺ concentration, and up-regulated the expression of Pmca2, a plasma membrane Ca²⁺ ATPase that transports intracellular Ca²⁺ ions to the extracellular compartment (FIGS. 12a and 12b).

Additionally, immunoblot analysis confirmed that, candesartan decreased ORAI2, STIM1, and TRPM7 protein levels, while increasing PMCA2 and SERCA2 protein levels compared to liver tissues of the control group; however, these increases were not statistically significant (FIG. 12c).

These results suggest that candesartan normalizes dysregulated intracellular Ca²⁺ homeostasis in HFD-fed mice by downregulating the expression of SOCE-related genes, including Orai2, Stim1, Trpc1, Trpm4, and Trpm7, or by upregulating the expression of Pmca2 and Serca2.

### Example 10. Analysis of Gene and Protein Expression Related to ER Stress, Glucose Metabolism, and Lipid Metabolism by Candesartan

Disruption of Ca²⁺ homeostasis in obesity is an important cause of ER stress, impaired protein folding, and dysregulation of carbohydrate and lipid metabolism. As candesartan normalizes dysregulated intracellular Ca²⁺ homeostasis in the liver of HFD-fed mice, the expression of genes and essential transcription factors associated with ER stress, glucose metabolism, and lipid metabolism was evaluated.

As a result, as shown in FIG. 13, candesartan dramatically reduced the expression of Atf3 and Xbp1, which are key transcription factors for ER stress and unfolded protein response (UPR) (FIG. 13a), and candesartan-mediated attenuation of intracellular Ca²⁺ overload significantly reduced the expression levels of many transcription factors for lipid metabolism, including Nur77, Nurr1, Ppard, and Pparg (FIG. 13b).

Consistent with these results, immunoblot analysis confirmed that candesartan significantly reduced the levels of NURR1, PPARg, and XBP1 compared to the control group (FIGS. 13c and 13d).

These results suggest that normalization of intracellular Ca²⁺ homeostasis is an important component of physiological and metabolic homeostasis.

### Example 11. Improvement of Obesity-Induced Insulin Resistance by Candesartan

To elucidate the role of candesartan on insulin signaling in vivo, mice were fasted overnight and then re-fed an HFD for 4 hours, and postprandial phosphorylation of AKT and its downstream signaling substrates FOXO1A, FOXO3A, GSK3B, and AS160 was measured.

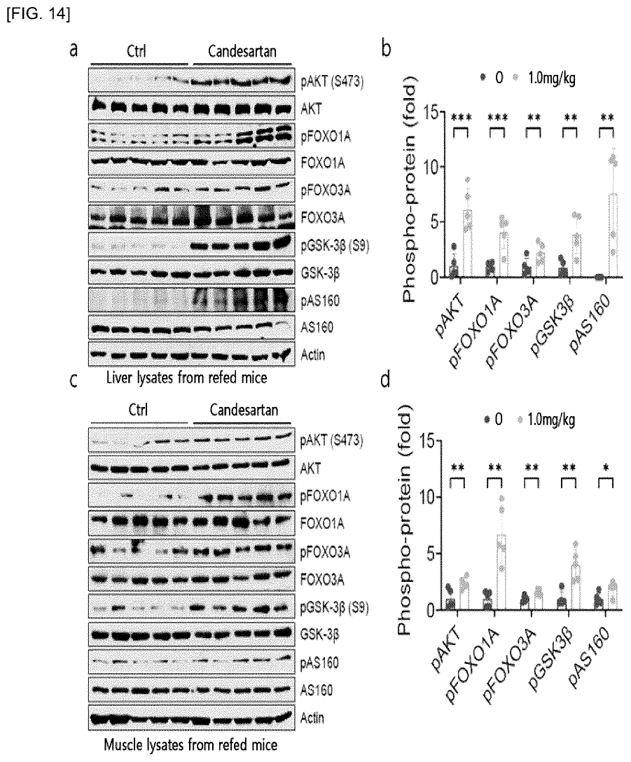

As shown in FIG. 14, the results were consistent with the in vitro effects. Candesartan dramatically increased the postprandial phosphorylation of AKT and its downstream signaling molecules FOXO1A, FOXO3A, GSK3B, and AS160 by 4- to 10-fold or more compared to the control group, thereby confirming that candesartan completely rescued the impaired insulin signaling of HFD-fed mice (FIGS. 14a and 14b). Similarly, candesartan administration was found to improve postprandial insulin signaling in muscle tissues (FIGS. 14c and 14d).

These results showed that candesartan, a drug that inhibits palmitic acid-induced intracellular Ca²⁺ overload in HFD-fed mice, substantially reversed obesity-induced insulin resistance by improving postprandial insulin signaling.

### Example 12. Evaluation of the Effect of Candesartan on AKT PH Domain Membrane Localization

To investigate whether candesartan regulates membrane localization of the AKT PH domain, the intracellular localization of adenoviral AKT PH mCherry vector as a biosensor for PI(3, 4, 5)P₃ was determined in mice fed HFD containing vehicle (control) or 1.0 mg/kg/day candesartan.

Specifically, AKT PH mCherry adenovirus (5×10⁸ plaque forming units) was delivered via systemic tail vein injection into C57BL/6J mice after 8 weeks of HFD feeding and 2 weeks of treatment with 1.0 mg/kg candesartan or vehicle. Seven days after adenovirus infection, livers were obtained from mice after overnight fasting and subsequent re-feeding with an HFD for 4 hours. For immunofluorescence, formalin-fixed liver sections or cells were plated on glass slides and counterstained with DAPI to visualize nuclei. After mounting, the sections were imaged with an LSM 700 laser scanning confocal microscope (Carl Zeiss), and the images were analyzed using ZEN Software (Carl Zeiss). Intracellular Ca²⁺ was quantified using the NIH ImageJ software (https://imagej.nih.gov/ij/, 1997-2017).

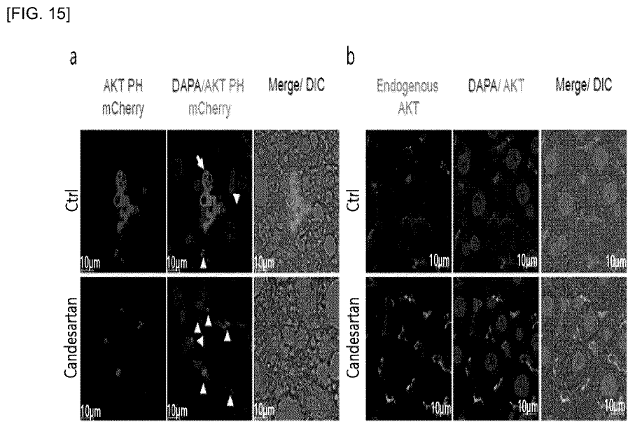

As a result, as shown in FIG. 15, adenovirus AKT PH mCherry was mainly localized to the cytoplasm, but some punctate adenovirus AKT PH mCherry was localized to the plasma membrane in hepatocytes of HFD-fed mice in response to re-feeding (FIG. 15a).

Subsequently, the intracellular localization of endogenous AKT was monitored in the livers of mice fed an HFD containing vehicle or 1.0 mg/kg/day candesartan. As a result, in hepatocytes of mice fed an HFD without candesartan, endogenous AKT was partially localized to the plasma membrane in response to re-feeding (FIG. 15b). In contrast, in hepatocytes of mice fed an HFD containing 1.0 mg/kg candesartan, endogenous AKT was predominantly accumulated at the plasma membrane in response to re-feeding, indicating that candesartan enhances insulin sensitivity by promoting postprandial membrane localization of endogenous AKT.

These results provide direct evidence that candesartan, a drug that inhibits obesity-related intracellular Ca²⁺ overload, improves insulin sensitivity and signaling by enhancing postprandial membrane localization of the AKT PH domain, thereby improving obesity-induced insulin resistance.

From the above description, those skilled in the art to which the present invention pertains would understand that the present invention can be implemented in other specific forms without changing its technical concept or essential features. In this regard, the above-described embodiments should be construed as being exemplified and not limiting the present invention. The scope of the present invention should be interpreted to include all modifications or modified forms derived from the meaning and scope of the claims as set forth below, as well as equivalent concepts, rather than from the detailed description above.

The present invention was supported by the following national research and development projects funded by the Ministry of Science and ICT:
1. Project Unique Number: 1711161844
Project Number: 2021R1A5A2030333
Project Management Agency: National Research Foundation of Korea
Research Project: Group research support
Project Title: Research Center for Innate Immunity-Mediated Chronic

### Inflammatory Diseases

Contribution Rate: 50%
Performing Institution: Gachon University
Research Period: Mar. 1, 2022, to Feb. 28, 2023
2. Project Unique Number: 1711174636
Project Number: 2022R1A2C2092700
Project Management Agency: National Research Foundation of Korea
Research Project: Individual basic research
Project Title: Identification of Novel Functions of PB1 Domain Proteins as Factors
Exacerbating Insulin Resistance
Contribution Rate: 50%
Performing Institution: Gachon University
Research Period: Sep. 1, 2022, to Feb. 28, 2023

1. A method for preventing or treating a metabolic disease, comprising administering a composition, which comprises candesartan or azilsartan to a subject.

2. The method of claim 1, wherein the candesartan comprises candesartan cilexetil and candesartan cilexetil related compound G.

3. The method of claim 1, wherein the azilsartan comprises azilsartan medoxomil.

4. The method of claim 1, wherein the metabolic disease is at least one selected from the group consisting of insulin resistance disease, hypertension, diabetes, obesity, fatty liver disease, dyslipidemia, liver disease, gout, kidney disease, and hyperuricemia.

5. The method of claim 1, wherein the composition exhibits an effect of improving insulin resistance.

6. The method of claim 5, wherein the improvement of insulin resistance is achieved by restoring impaired insulin signaling through normalization of intracellular Ca²⁺, inhibition of lipid accumulation, and rescue of intracellular localization of a PH domain.

7. The method of claim 6, wherein the normalization of intracellular Ca²⁺ is achieved by regulating the expression of essential Ca²⁺ channels or transporters involved in regulating intracellular Ca²⁺ concentration.

8. The method of claim 7, wherein the normalization of intracellular Ca²⁺ is the normalization of calcium concentration in the endoplasmic reticulum.

9. The method of claim 7, wherein the regulation of transporter expression is downregulating the expression of a store-operated channel (SOC)-related gene.

10. The method of claim 2, wherein the SOC-related gene is inositol 1,4,5-trisphosphate receptor type 3(Itpr3), Orai2, stromal interaction molecule 1(Stim1), and transient receptor potential canonical 1(Trpc1).

11. The method of claim 1, wherein the composition exhibits an effect of improving fatty liver.

12. The method of claim 11, wherein the improvement of fatty liver is achieved by reducing hepatic triglyceride content, ALT, AST, triglycerides, and low-density lipoprotein cholesterol levels, infiltration of inflammatory macrophages, and liver inflammation.

13. The method of claim 1, wherein the composition further exhibits an effect of improving kidney function or an effect of improving gout.

14. The method of claim 13, wherein the improvement of kidney function or gout is achieved by reducing creatinine or uric acid levels in serum.

15. (canceled)

16. (canceled)
